SEQUENCE LISTING (23 sequences)

SEQ ID NO: 1 - DNA, 321 bp, Homo sapiens
gacatccagatgacccagtctccatcttccgtgtctgcatctataggagacagagtcaccatcacttgtcgggcgagtcagggtattgacaactggttaggctggtatcagcagaaacctgggaaagcccctaaactcctgatctacgatgcatccaatttggacacaggggtcccatcaaggttcagtggaagtggatctgggacatattttactctcaccatcagtagcctgcaagctgaagattttgcagtttatttctgtcaacaggctaaagcttttcctcccactttcggcggagggaccaaggtggacatcaaa

SEQ ID NO: 2 - protein, 107 aa, Homo sapiens [sequence omitted]

SEQ ID NO: 3 - DNA, 348 bp, Homo sapiens
gaggtccagctggtgcagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagtcacagatgcttttgatatctggggccaagggacaatggtcaccgtctcaagc

SEQ ID NO: 4 - protein, 116 aa, Homo sapiens [sequence omitted]

SEQ ID NO: 5 - DNA, 642 bp, Homo sapiens
gacatccagatgacccagtctccatcttccgtgtctgcatctataggagacagagtcaccatcacttgtcgggcgagtcagggtattgacaactggttaggctggtatcagcagaaacctgggaaagcccctaaactcctgatctacgatgcatccaatttggacacaggggtcccatcaaggttcagtggaagtggatctgggacatattttactctcaccatcagtagcctgcaagctgaagattttgcagtttatttctgtcaacaggctaaagcttttcctcccactttcggcggagggaccaaggtggacatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 6 - protein, 214 aa, Homo sapiens
DIQMTQSPSSVSASIGDRVTITCRASQGIDNWLGWYQQKPGKAPKLLIYDASNLDTGVPSRFSGSGSGTYFTLTISSLQAEDFAVYFCQQAKAFPPTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - DNA, 1338 bp, Homo sapiens
gaggtccagctggtgcagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagtcacagatgcttttgatatctggggccaagggacaatggtcaccgtctcaagcgctagcaccaagggcccatcggtcctccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 8 - protein, 446 aa, Homo sapiens
EVQLVQSGGGLVKPGGSLRLSCAASGFTFSSYSMNWVRQAPGKGLEWVSSISSSSSYIYYADSVKGRFTISRDNAKNSLYLQMNSLRAEDTAVYYCARVTDAFDIWGQGTMVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 1337 aa, Homo sapiens
ASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 10 - DNA, 324 bp, Artificial Sequence
  synthetic construct
gacatccagatgacccagtctccatcctccctgtctgcatctgtaggagacagagtcaccatcacttgcagtgtcagctcaagtgtatcctccatttacttgcactggtatcagcagaaaccagggaaagcccctaagctcctgatctatagcacatccaacttggcttctggagtcccatcaaggttcagtggcagtggatctgggacagatttcactctcaccatcagcagtctgcaacctgaagattttgcaacttactactgtcaagtctacagtggttacccgctcacgttcggcggagggaccaaggtggagatcaaa

SEQ ID NO: 11 - protein, 108 aa, Artificial Sequence [sequence omitted]

SEQ ID NO: 12 - DNA, 342 bp, Artificial Sequence
  synthetic construct
caggttcagctggtgcagtctggtgctgaggtgaagaagcctggtgcctcagtgaaggtctcctgcaaggcttctggttacacctttaccgactactacatgcactgggtgcgtcaggcccctggtcaaggtcttgagtggatgggtcgtgttaatcctaaccggaggggtactacctacaaccagaaattcgagggccgtgtcaccatgaccacagacacatccacgagcacagcctacatggagctgcgtagcctgcgttctgacgacacggccgtgtattactgtgcgcgtgcgaactggcttgactactggggccagggcaccaccgtcaccgtctcc

SEQ ID NO: 13 - protein, 115 aa, Artificial Sequence [sequence omitted]

SEQ ID NO: 14 - DNA, 651 bp, Artificial Sequence
  synthetic construct
gacatccagatgacccagtctccatcctccctgtctgcatctgtaggagacagagtcaccatcacttgcagtgtcagctcaagtgtatcctccatttacttgcactggtatcagcagaaaccagggaaagcccctaagctcctgatctatagcacatccaacttggcttctggagtcccatcaaggttcagtggcagtggatctgggacagatttcactctcaccatcagcagtctgcaacctgaagattttgcaacttactactgtcaagtctacagtggttacccgctcacgttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgctaatag

SEQ ID NO: 15 - protein, 215 aa, Artificial Sequence
  synthetic construct
DIQMTQSPSSLSASVGDRVTITCSVSSSVSSIYLHWYQQKPGKAPKLLIYSTSNLASGVPSRFSGSGSGTDFTLTISSLQPEDFATYYCQVYSGYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - DNA, 1323 bp, Artificial Sequence
  synthetic construct
caggttcagctggtgcagtctggtgctgaggtgaagaagcctggtgcctcagtgaaggtctcctgcaaggcttctggttacacattcactgactactacatgcactgggtgcgtcaggcccctggtcaaggtcttgagtggatgggtcgtgttaatcctaaccggaggggtactacctacaaccagaaattcgagggccgtgtcaccatgaccacagacacatccacgagcacagcctacatggagctgcgtagcctgcgttctgacgacacggccgtgtattactgtgcgcgtgcgaactggcttgactactggggccagggcaccaccgtcaccgtctcctccgcctccaccaagggcccatcggtcttcccgctagcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgaggccgccgggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggaaagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 17 - protein, 441 aa, Artificial Sequence
  synthetic construct
QVQLVQSGAEVKKPGASVKVSCKASGYTFTDYYMHWVRQAPGQGLEWMGRVNPNRRGTTYNQKFEGRVTMTTDTSTSTAYMELRSLRSDDTAVYYCARANWLDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 18 - protein, 932 aa, Artificial Sequence
  synthetic construct
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFT

SEQ ID NO: 19 - protein, 908 aa, Homo sapiens
ECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFT

SEQ ID NO: 20 - protein, 283 aa, Homo sapiens
ECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKR

SEQ ID NO: 21 - protein, 212 aa, Homo sapiens
STKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLG

SEQ ID NO: 22 - protein, 214 aa, Artificial Sequence
  synthetic construct
EIQMTQSPASLSASVGETVTITCRASENIYSYLAWYQRKQGRSPQLLVYNAKPLAEGVPSRFSGSGSGTQFSLKINSLQPEDFGTYYCQHHYGTPFTFGSGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 23 - protein, 443 aa, Artificial Sequence
  synthetic construct
EVQLQQSGTVLARPGASVKMSCKASGYSFTSYWMYWVKQRPGQGLEWIGGFHPRNSGTNYNQKFKGKAKLTAVTSASTAYMELSSLTNEDSAVYYCTRGYYYDGSFTYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

(19) United States
(12) Patent Application Publication
(10) Pub. No.: US 2016/0369004 A1
(43) Pub. Date: Dec. 22, 2016

(54) ANTI-MET IN COMBINATION WITH ANTI-VEGFR2 ANTIBODIES THERAPY FOR CANCER

(71) Applicant: Eli Lilly and Company, Indianapolis, IN (US)

(72) Inventors: Bronislaw Pytowski, New York, NY (US); Jonathan David Schwartz, Montclair, NJ (US); Volker Wacheck, Vienna (AT); Sau-Chi Betty Yan, Indianapolis, IN (US)

(73) Assignee: Eli Lilly and Company, Indianapolis, IN (US)

(21) Appl. No.: 15/122,262

(22) PCT Filed: Feb. 25, 2015
(86) PCT No.: PCT/US15/17424

§ 371 Date: Aug. 29, 2016

Related U.S. Application Data
(60) Provisional application No. 61/947,743, filed on Mar. 4, 2014.

(51) Int. Cl. C07K 16/30; A61K 45/06; C07K 16/28

(52) U.S. Cl. CPC C07K 16/30; C07K 16/2863; A61K 45/06; C07K 16/3046; A61K 2039/507

(57) ABSTRACT

The present invention provides preparation of medicaments for use in treating and methods of treating cancer selected from the group consisting of gastric cancer, HCC, and RCC comprising a C8-H241 Ab, preferably, C8-H241-IgG4, more preferably, emibetuzumab, in combination, as described herein, with an anti-VEGFR2 Ab, preferably, ramucirumab.

The present invention relates to combinations of an anti-human MET neutralizing and internalizing bivalent antibody, preferably, C8-H241-IgG4, more preferably, emibetuzumab, with an anti-human VEGFR-2 antibody, preferably, ramucirumab, and to methods of using the combinations to treat certain disorders, such as hepatocellular carcinoma, renal cell carcinoma, gastric cancer, preferably, carcinoma of the gastroesophageal junction, and lung cancer, preferably, non-small cell lung cancer.

The present invention is in the field of treatment of cancer.

Hepatocellular carcinoma (HCC) is the most common type of liver cancer. Most cases of HCC are secondary to either a viral hepatitis infection (hepatitis B or C) or cirrhosis (alcoholism being the most common cause of hepatic cirrhosis). Treatment options for HCC and prognosis are dependent on many factors but especially on tumor size, grade, and staging.

Renal cell carcinoma or renal cell cancer (RCC) is a kidney cancer that originates in the very small tubes in the kidney that transport glomerular filtrate from the glomerulus to the descending limb of the nephron. RCC is the most common type of kidney cancer in adults and is responsible for approximately 80% of kidney cancer cases. It is among the most lethal of all urological cancers. Initial treatment is typically a radical or partial nephrectomy and remains the mainstay of curative treatment. Where the tumor is confined to the renal parenchyma, the 5-year survival rate is 60-70%, but this is lowered considerably when it has metastasized. It is relatively resistant to radiation therapy and chemotherapy, although some cases respond to immunotherapy.

Gastric cancer is a malignant tumor that originates in the stomach lining. Gastric cancers are classified according to the type of tissue from which they originate, with the most common type being adenocarcinoma, which starts in the glandular tissue of the stomach and accounts for over 90% of all stomach cancers. Adenocarcinoma of the esophagus including carcinoma of the gastroesophageal junction (GEJ), is one of the fastest rising malignancies and is associated with a poor prognosis. Other forms of gastric cancer include lymphomas and sarcomas. Gastric cancer may be cured if it is found and treated at an early stage, but unfortunately, it is often found at a later stage.

Lung cancer ranks as one of the most common causes of death due to cancer in both men and women throughout the world. The two main types of lung cancer are small cell lung cancer and non-small cell lung cancer (NSCLC). Non-small cell lung cancer accounts for approximately 85% of all lung cancers. Disease stage is the primary consideration for treatment of NSCLC. When feasible, surgical resection is the treatment of choice for early-stage localized disease, whereas patients with locally advanced disease frequently require multimodality therapy. The majority of patients with lung cancer have advanced and/or metastatic disease at diagnosis and the majority of patients treated with curative intent develop recurrence. These patients present with advanced, inoperable stage cancer for which there is no prospect of cure. Treatment is provided to improve symptoms, optimize quality of life, and prolong survival.

Unfortunately, a cure for these cancers still remains elusive and there exists a need for more and different therapies that may prove to be effective in treating them.

Emibetuzumab is an affinity-optimized, humanized and bivalent anti-human MET monoclonal antibody previously disclosed in WO 2010/059654. Emibetuzumab has high neutralization and internalization activities resulting in inhibition of both HGF-dependent and HGF-independent MET pathway activation and tumor growth. Emibetuzumab exhibits no functional agonist activity in multiple cell-based assays (Liu, L., et al., LY2875358, a Neutralizing and Internalizing Anti-MET Bivalent Antibody, Inhibits HGF-Dependent and HGF-Independent MET Activation and Tumor Growth. Clinical Cancer Research, 20; 6059 (December 2014)). Moreover, this unique bivalent anti-human MET monoclonal antibody has potent antitumor activity in both HGF-dependent and HGF-independent (e.g., MET amplified) xenograft tumor models (Liu, L., et al., (2014). Furthermore, clinical activity for the antibody has been observed in patients with advanced cancer (Goldman, J W, et al. First-in-human dose escalation study of LY2875358, a bivalent MET antibody, as monotherapy and in combination with erlotinib in patients with advanced cancer. American Society of Clinical Oncology (ASCO) Annual Meeting (2013); poster 115545). Emibetuzumab is currently being evaluated in Phase 2 clinical studies in combination with erlotinib in NSCLC patients (see, ClinicalTrials.gov NCT01900652, NCT01897480).

Ramucirumab is a fully human monoclonal antibody directed against the human vascular endothelial growth factor receptor 2 (VEGFR2). Ramucirumab and methods of making and using this compound including for the treatment of neoplastic diseases such as solid and non-solid tumors are disclosed in WO 2003/075840. Furthermore, clinical activity for ramucirumab has also been reported in patients with several cancer types including gastric and GEJ, as well as HCC, NSCLC, and RCC. On April, 2014 and Dec. 16, 2014, ramucirumab (Cyramza®) was approved by the U.S. Food and Drug Administration (FDA) for treating gastric cancer (or carcinoma of the GEJ) and NSCLC, respectively.

A novel combination of a C8-H241 Ab, preferably, C8-H241-IgG4, more preferably, emibetuzumab, and an anti-VEGFR2 Ab, preferably, ramucirumab, is herein presented. Although combinations of MET inhibitors and inhibitors of VEGF and combinations of MET inhibitors and inhibitors of VEGF receptors have been contemplated in the art (see, for example, WO 2011/143665 and WO 2012/044577, respectively), the present inventors disclose herein methods of treating cancer by using a novel combination of a C8-H241 Ab and an anti-VEGFR2 Ab as part of a specific treatment regimen that provides unexpected beneficial therapeutic effects from the combined activity of these therapeutic agents in some cancer patients as compared to the therapeutic effects provided by either agent alone. The present inventors also disclose herein methods of treating cancer by using a novel combination of C8-H241-IgG4, preferably, emibetuzumab, and ramucirumab as part of a specific treatment regimen that provides unexpected beneficial therapeutic effects from the combined activity of these therapeutic agents in some cancer patients as compared to the therapeutic effects provided by either agent alone. Preferably, the cancer is gastric, HCC, RCC, or lung cancer. More preferably, the cancer is gastric cancer or carcinoma of the GEJ, and optionally the treatment regimen includes paclitaxel and/or a combination of 5-fluorouracil, folinic acid and oxaliplatin. Even more preferably, the cancer is HCC and, optionally, the treatment regimen includes a combination of 5-fluorouracil, folinic acid and oxaliplatin. Even more preferably, the cancer is RCC and, optionally, the treatment regimen includes everolimus or temsirolimus. Even more preferably, the cancer is lung cancer and, optionally, the treatment regimen includes docetaxel, pemetrexed, gemcitabine, or one or more EGFR inhibitors such as erlotinib, gefitinib, afatinib, rociletinib, AZD9291, ASP8273, or HM61713. Even more preferably, the cancer is NSCLC, and, optionally, the treatment regimen includes docetaxel, pemetrexed, gemcitabine or one or more EGFR inhibitors such as erlotinib, gefitinib, afatinib, rociletinib, AZD9291, ASP8273, or HM61713.

Accordingly, the present invention provides a method of treating cancer in a patient, comprising administering to a cancer patient in need of such treatment an effective amount of a C8-H241 Ab in combination with an effective amount of an anti-VEGFR2 Ab. The present invention also provides a method of treating cancer in a patient, comprising administering to a cancer patient in need of such treatment an effective amount of C8-H241-IgG4, preferably, emibetuzumab, in combination with ramucirumab. Optionally, these methods further comprise the administration of an effective amount of one or more anti-tumor agents selected from the group consisting of paclitaxel, docetaxel, pemetrexed, gemcitabine, everolimus, temsirolimus, one or more EGFR inhibitors such as erlotinib, gefitinib, afatinib, rociletinib, AZD9291, ASP8273, and HM61713, or a pharmaceutically acceptable salt thereof, and/or a combination of 5-fluorouracil, folinic acid and oxaliplatin. An effective amount of these anti-tumor agents is typically the dose stated on that agents label. Preferably, the cancer in the aforementioned methods is gastric cancer, and more preferably, carcinoma of the gastroesophageal junction, and optionally these methods also include administering an effective amount of paclitaxel and/or a combination of 5-fluorouracil, folinic acid and oxaliplatin. Even more preferably, the cancer in the aforementioned methods is HCC and optionally these methods also include administering an effective amount of a combination of 5-fluorouracil, folinic acid and oxaliplatin. Even more preferably, the cancer in the aforementioned methods is RCC and optionally these methods also include administering an effective amount of everolimus or temsirolimus. Even more preferably, the cancer in the aforementioned methods is lung cancer and, optionally, the treatment regimen includes docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof. Even more preferably, the cancer in the aforementioned methods is NSCLC and, optionally, the treatment regimen includes docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof.

The invention further provides a pharmaceutical composition comprising a C8-H241 Ab with one or more pharmaceutically acceptable carriers, diluents, or excipients, in combination with a pharmaceutical composition of an anti-VEGFR2 Ab with one or more pharmaceutically acceptable carriers, diluents, or excipients. The invention also provides a pharmaceutical composition comprising C8-H241-IgG4, preferably, emibetuzumab, with one or more pharmaceutically acceptable carriers, diluents, or excipients, in combination with a pharmaceutical composition of ramucirumab, with one or more pharmaceutically acceptable carriers, diluents, or excipients. Optionally, a combination for use in the treatment of gastric cancer, preferably, carcinoma of the GEJ further comprises paclitaxel and/or a combination of 5-fluorouracil, folinic acid and oxaliplatin. Optionally, a combination for use in the treatment of HCC further comprises a combination of 5-fluorouracil, folinic acid and oxaliplatin. Optionally, a combination for use in the treatment of RCC further comprises everolimus or temsirolimus. Optionally, a combination for use in the treatment of lung cancer and/or NSCLC further comprises docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof.

In addition, the invention provides a kit comprising a C8-H241 Ab and an anti-VEGFR2 Ab. The invention also provides a kit comprising C8-H241-IgG4, preferably, emibetuzumab, and ramucirumab. The invention further provides a kit comprising a pharmaceutical composition comprising a C8-H241 Ab with one or more pharmaceutically acceptable carriers, diluents, or excipients, and a pharmaceutical composition comprising an anti-VEGFR2 Ab, with one or more pharmaceutically acceptable carriers, diluents, or excipients. The invention also provides a kit comprising a pharmaceutical composition comprising C8-H241-IgG4, preferably, emibetuzumab, with one or more pharmaceutically acceptable carriers, diluents, or excipients, and a pharmaceutical composition comprising ramucirumab, with one or more pharmaceutically acceptable carriers, diluents, or excipients. Optionally, the kit also includes compositions comprising at least one of paclitaxel, everolimus, temsirolimus, docetaxel, pemetrexed, gemcitabine, 5-fluorouracil, folinic acid, oxaliplatin or an EGFR inhibitor such as erlotinib, gefitinib, afatinib, rociletinib, AZD9291, ASP8273, HM61713, or a pharmaceutically acceptable salt thereof.

The invention further provides a combination comprising a C8-H241 Ab and an anti-VEGFR2 Ab, for simultaneous, separate or sequential use in the treatment of gastric cancer, preferably, carcinoma of the GEJ. The invention further provides a combination comprising C8-H241-IgG4, preferably, emibetuzumab, and ramucirumab, for simultaneous, separate or sequential use in the treatment of gastric cancer, preferably, carcinoma of the GEJ. Optionally, these combinations further comprise paclitaxel, a combination of 5-fluorouracil, folinic acid and oxaliplatin, or a pharmaceutically acceptable salt thereof.

The invention further provides a combination comprising a C8-H241 Ab and an anti-VEGFR2 Ab, for simultaneous, separate or sequential use in the treatment of HCC. The invention further provides a combination comprising C8-H241-IgG4, preferably, emibetuzumab, and ramucirumab, for simultaneous, separate or sequential use in the treatment of HCC. Optionally, these combinations further comprise a combination of 5-fluorouracil, folinic acid and oxaliplatin.

The invention further provides a combination comprising a C8-H241 Ab and an anti-VEGFR2 Ab, for simultaneous, separate or sequential use in the treatment of RCC. The invention further provides a combination comprising C8-H241-IgG4, preferably, emibetuzumab, and ramucirumab, for simultaneous, separate or sequential use in the treatment of RCC. Optionally, these combinations further comprise everolimus or temsirolimus.

The invention further provides a combination comprising a C8-H241 Ab and an anti-VEGFR2 Ab, for simultaneous, separate or sequential use in the treatment of NSCLC. The invention further provides a combination comprising C8-H241-IgG4, preferably, emibetuzumab, and ramucirumab, for simultaneous, separate or sequential use in the treatment of NSCLC. Optionally, these combinations further comprise docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof.

The invention further provides a combination of a C8-H241 Ab and an anti-VEGFR2 Ab for use in therapy. The invention further provides a combination of C8-H241-IgG4 and ramucirumab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin are also included in the combination therapy.

The invention further provides the use of a combination of a C8-H241 Ab and an anti-VEGFR2 Ab for the manufacture of a medicament for the treatment of gastric cancer, preferably, carcinoma of the GEJ. The invention further provides the use of a combination of C8-H241-IgG4, preferably, emibetuzumab, and ramucirumab for the manufacture of a medicament for the treatment of gastric cancer, preferably, carcinoma of the GEJ. Optionally, these combinations further comprise use of paclitaxel and/or a combination of 5-fluorouracil, folinic acid and oxaliplatin.

The invention further provides the use of a combination of a C8-H241 Ab and an anti-VEGFR2 Ab for the manufacture of a medicament for the treatment of HCC. The invention further provides the use of a combination of C8-H241-IgG4, preferably, emibetuzumab, and ramucirumab for the manufacture of a medicament for the treatment of HCC. Optionally, these combinations further comprise use of a combination of 5-fluorouracil, folinic acid and oxaliplatin.

The invention further provides the use of a combination of a C8-H241 Ab and an anti-VEGFR2 Ab for the manufacture of a medicament for the treatment of RCC. The invention further provides the use of a combination of C8-H241-IgG4, preferably, emibetuzumab, and ramucirumab for the manufacture of a medicament for the treatment of RCC. Optionally, these combinations further comprise use of everolimus or temsirolimus.

The invention further provides the use of a combination of a C8-H241 Ab and an anti-VEGFR2 Ab for the manufacture of a medicament for the treatment of NSCLC. The invention further provides the use of a combination of C8-H241-IgG4, preferably, emibetuzumab, and ramucirumab for the manufacture of a medicament for the treatment of NSCLC. Optionally, these combinations further comprise use of paclitaxel, docetaxel, pemetrexed, gemcitabine, or one or more EGFR inhibitors such as erlotinib, gefitinib, afatinib, rociletinib, AZD9291, ASP8273, HM61713, or a pharmaceutically acceptable salt thereof.

Another aspect of the present invention is a method of treating gastric cancer, carcinoma of the GEJ, HCC, RCC, lung cancer, or NSCLC in a patient afflicted therewith, comprising administering to a cancer patient in need of treatment:
a) ramucirumab at 8 mg/kg on days 1 and 15 of a 28-day cycle; and
b) C8-H241-IgG4 at 750 mg on days 1 and 15 of the 28-day cycle of step (a).

Another aspect of the present invention is a method of treating gastric cancer, carcinoma of the GEJ, HCC, RCC, lung cancer, or NSCLC in a patient afflicted therewith, comprising administering to a cancer patient in need of treatment:
a) ramucirumab at 8 mg/kg on days 1 and 15 of a 28-day cycle; and
b) emibetuzumab at 750 mg on days 1 and 15 of the 28-day cycle of step (a).

A further aspect of the present invention provides:
a) use of ramucirumab for the manufacture of a medicament for the treatment of gastric cancer, carcinoma of the GEJ, HCC, RCC, lung cancer, or NSCLC; and
b) use of C8-H241-IgG4 for the manufacture of a medicament for the treatment of gastric cancer, carcinoma of the GEJ, HCC, RCC, lung cancer, or NSCLC,

wherein ramucirumab is administered at 8 mg/kg on days 1 and 15 of a 28-day cycle and C8-H241-IgG4 is administered at 750 mg on days 1 and 15 of the same 28-day cycle.

A further aspect of the present invention provides:
a) use of ramucirumab for the manufacture of a medicament for the treatment of gastric cancer, carcinoma of the GEJ, HCC, RCC, lung cancer, or NSCLC; and
b) use of emibetuzumab for the manufacture of a medicament for the treatment of gastric cancer, carcinoma of the GEJ, HCC, RCC, lung cancer, or NSCLC, wherein ramucirumab is administered at 8 mg/kg on days 1 and 15 of a 28-day cycle and emibetuzumab is administered at 750 mg on days 1 and 15 of the same 28-day cycle.

As used herein, the term “anti-VEGFR2 Ab” refers to an antibody comprising: a light chain variable region (LCVR) whose amino acid sequence is that given in SEQ ID NO: 2, and a heavy chain variable region (HCVR) whose amino acid sequence is that given in SEQ ID NO: 4, wherein the antibody specifically binds to VEGFR2-ECD. In some embodiments, an anti-VEGFR2 Ab is an antibody comprising: a light chain (LC) whose amino acid sequence is that given in SEQ ID NO: 6, and a heavy chain (HC) whose amino acid sequence is that given in SEQ ID NO: 8 and wherein the antibody specifically binds to VEGFR2-ECD. In other embodiments of the present invention the anti-VEGFR2 Ab is ramucirumab. The anti-VEGFR2 Ab selected will generally bind VEGFR2-ECD with a K_{D} value of between about 100 nM to about 1 pM. Antibody affinities may be determined by a SPR based assay (such as the BIAcore assay as described in PCT Application Publication No. WO 2005/012359); enzyme-linked immunoabsorbent assay (ELISA); and competition assays (e.g., a radiolabeled antigen binding assay (RIA)), for example.

As used herein, the term “AZD9291” refers to an orally available, irreversible EGFR inhibitor (see, for example, CAS registry numbers 1421373-65-0, 1421373-66-1 (mesylate salt)), that is under development for the treatment of patients with metastatic EGFR mutation-positive (such as L858R, exon 19 deletion and T790M) NSCLC.

As used herein, the term “ASP8273” refers to an orally available, irreversible, mutant-selective, EGFR inhibitor, with potential antineoplastic activity, that is under development for the treatment of patients with NSCLC with EGFR mutations. Upon oral administration, ASP8273 covalently binds to and inhibits the activity of mutant forms of EGFR, including the T790M EGFR mutant (see, for example, Sakagami et al., AACR Annual Meeting April 2014, abstract 1728, or PCT Application Publication No. WO 2013108754).

As used herein, the term “HM61713” refers to an orally active, EGFR mutant selective inhibitor with anti-cancer activity in several EGFR mutant lung cancer cell lines including T790M mutation harboring cell lines and under development for the treatment of patients with NSCLC with EGFR mutations who failed prior EGFR tyrosine kinase inhibitor therapy (see, for example, Kim D et al., J Clin Oncol 2014; 32(Suppl):abstract 8011; or PCT Application Publication No. WO 2014140989).

As used herein, the term “rocelitinib” refers to an orally available, irreversible EGFR inhibitor (see, for example, CAS registry numbers 1374640-70-6 (free base), 1446700-26-0 (hydrobromide salt)), that is under development for the treatment of patients with metastatic EGFR mutation-positive (such as T790M mutation) NSCLC (see, for example, Sequist, et al., 2014 American Society of Clinical Oncology (ASCO) Annual Meeting; May 2014, Abstract #8010). Rociletinib, is also known in the art as AVL-301 and CO-1686.

Each of the embodiments described herein envisions within its scope pharmaceutically acceptable salts of the compounds referenced or described herein. Accordingly, the phrase “or a pharmaceutically acceptable salt thereof” is implicit in the references to or descriptions of all compounds herein.

The term “K_{D}”, as used herein, is intended to refer to the equilibrium dissociation constant of a particular antibody-antigen or antibody fragment-antigen interaction.

The phrase “specifically binds” as used herein in reference to the affinity of a antibody, or antigen-binding fragment thereof, for the MET-ECD or VEGFR2-ECD is intended to mean, unless indicated otherwise, a K_{D} of less than about 1×10⁻⁸ M, preferably, less than about 1×10⁻⁹ M as determined by common methods known in the art, including by use of a surface plasmon resonance (SPR) biosensor at 25° C. (for MET-ECD) or 37° C. (for VEGFR2-ECD).

As used herein, the term “ramucirumab”, also known as Cyramza®, IMC-1121b, and/or CAS registry number 947687-13-0, refers to an anti-VEGFR2 Ab comprising: two light chains, each of whose amino acid sequence is that given in SEQ ID NO: 6, and two heavy chains, each of whose amino acid sequence is that given in SEQ ID NO: 8.

In certain embodiments, the anti-VEGFR2 Ab, preferably, ramucirumab, binds the extracellular domain of VEGFR2 (i.e., VEGFR2-ECD) with a K_{D} value of between about 100 nM to about 1 pM, preferably, between about 10 nM to about 10 pM, more preferably, between about 10 nM to about 100 pM, more preferably, between about 5.0 nM to about 0.5 nM, more preferably between about 5.0 nM and 2 nM, and most preferably about 3.5 nM, as determined by a SPR based assay (such as the BIAcore assay as described in PCT Application Publication No. WO 2005/012359) conducted at 37° C.

As used herein, the term “VEGFR2” refers to the polypeptide whose amino acid sequence is that given in SEQ ID NO: 9. VEGFR2 is also known as kinase domain receptor (KDR). Unless stated otherwise, the term “VEGFR2-ECD” as used herein means the protein beginning and ending at amino acids 1 and 744, respectively, of SEQ ID NO: 9.

As used herein, the term “C8-H241 Ab” refers to refers to an antibody comprising: a LCVR whose amino acid sequence is that given in SEQ ID NO: 11, and a HCVR whose amino acid sequence is that given in SEQ ID NO: 13, wherein that the C8-H241 Ab specifically binds to MET-ECD. In some embodiments, the C8-H241 Ab is an antibody comprising: a LC whose amino acid sequence is that given in SEQ ID NO: 15, and a HC whose amino acid sequence is that given in SEQ ID NO: 17 and that specifically binds to MET-ECD. In other embodiments of the present invention the C8-H241 Ab is C8-H241-IgG4, preferably, emibetuzumab.

As used herein, the term “C8-H241-IgG4” refers to a C8-H241 Ab comprising: two light chains, each of whose amino acid sequence is that given in SEQ ID NO: 15, and two heavy chains, each of whose amino acid sequence is that given in SEQ ID NO: 17.

As used herein, the term “emibetuzumab” refers to a C8-H241-IgG4 comprising: two light chains, each of whose amino acid sequence is that given in SEQ ID NO: 15, and two heavy chains, each of whose amino acid sequence is that given in SEQ ID NO: 17 (see, WHO Drug Information, Proposed International Nonproprietary Names (INN) List 111, Volume 28, No. 2, July 2014).

In certain embodiments, the C8-H241 Ab, preferably, C8-H241-IgG4, more preferably, emibetuzumab, binds MET-ECD with a K_{D} value of between about 100 nM to about 1 pM, preferably, between about 10 nM to about 10 pM, more preferably, between about 10 nM to about 100 pM, more preferably, between about 2.5 nM to about 0.5 nM, and most preferably about 1 nM, as determined by a surface plasmon resonance (SPR) based assay (such as the BIAcore assay as described in PCT Application Publication No. WO 2005/012359) conducted at 25° C. Antibody affinities may also be determined by ELISA; and competition assays (e.g., a RIA), for example.

The terms “MET polypeptide”, “MET receptor”, “MET”, “HGF receptor” or “HGFR” are used interchangeably herein and, unless otherwise indicated, are intended to refer to the human receptor tyrosine kinase, as well as functionally active, mutated forms thereof, that bind human hepatocyte growth factor. Specific examples of MET include, e.g., a human polypeptide encoded by the nucleotide sequence provided in GenBank accession no. NM_000245, or the human protein encoded by the polypeptide sequence provided in GenBank accession no. NP_000236.

The extracellular domain of MET has the amino acid sequence shown in, for example, SEQ ID NO: 18. However, amino acids 1-24 of SEQ ID NO: 18 comprise the signal sequence. Therefore, unless stated otherwise, the term “MET-ECD” as used herein means the protein beginning and ending at amino acids 25 and 932, respectively, of SEQ ID NO: 18 (i.e., SEQ ID NO: 19). The SEMA domain consists of approximately 500 amino acid residues at the N-terminus of MET, and contains the α-chain (amino acid residues 25-307 of SEQ ID NO: 18 (i.e., SEQ ID NO: 20) and part of the β-chain (amino acid residues 308-519 of SEQ ID NO: 18 (i.e., SEQ ID NO: 21)).

Unless indicated otherwise, the term “antibody” refers to an immunoglobulin molecule comprising two heavy chains and two light chains interconnected by disulfide bonds. The amino terminal portion of each chain includes a variable region of about 100 to about 110 amino acids primarily responsible for antigen recognition via the complementarity determining regions (CDRs) contained therein. The carboxy-terminal portion of each chain defines a constant region primarily responsible for effector function.

As used herein, the term “antigen-binding fragment” refers to any antibody fragment that retains the ability to bind to its antigen. Such “antigen-binding fragments” can be selected from the group consisting of Fv, scFv, Fab, F(ab′)₂, Fab′, scFv-Fc fragments and diabodies. An antigen-binding fragment of an antibody will typically comprise at least one variable region. Preferably, an antigen-binding fragment comprises a heavy chain variable region (HCVR) and a light chain variable region (LCVR). More preferably, an antigen-binding fragment as used herein comprises a HCVR and a LCVR which confers antigen-binding specificity to VEGFR2-ECD (i.e., a “VEGFR2-ECD binding fragment”) or MET-ECD (i.e., a “MET-ECD binding fragment”).

As used herein, the term “light chain variable region (LCVR)” refers to a portion of a LC of an antibody molecule that includes amino acid sequences of Complementarity Determining Regions (CDRs; i.e., LCDR1, LCDR2, and LCDR3), and Framework Regions (FRs).

As used herein, the term “heavy chain variable region (HCVR)” refers to a portion of a HC of an antibody molecule that includes amino acid sequences of Complementarity Determining Regions (CDRs; i.e., HCDR1, HCDR2, and HCDR3), and Framework Regions (FRs).

As used herein, the terms “complementarity determining region” and “CDR”, refer to the non-contiguous antigen combining sites found within the variable region of LC and HC polypeptides of an antibody or an antigen-binding fragment thereof. These particular regions have been described by others including Kabat, et al., Ann. NY Acad. Sci. 190:382-93 (1971); Kabat et al., J. Biol. Chem. 252:6609-6616 (1977); Kabat, et al., Sequences of Proteins of Immunological Interest, Fifth Edition, U.S. Department of Health and Human Services, NIH Publication No. 91-3242 (1991); Chothia, et al., J. Mol. Biol. 196:901-917 (1987); MacCallum, et al., J. Mol. Biol., 262:732-745 (1996); and North, et al., J. Mol. Biol., 406, 228-256 (2011), where the definitions include overlapping or subsets of amino acid residues when compared against each other.

The CDRs are interspersed with regions that are more conserved, termed framework regions (“FR”). Each LCVR and HCVR is composed of three CDRs and four FRs, arranged from amino-terminus to carboxy-terminus in the following order: PRE CDR1, FR2, CDR2, FR3, CDR3, FR4. The three CDRs of the light chain are referred to as “LCDR1, LCDR2, and LCDR3” and the three CDRs of the HC are referred to as “HCDR1, HCDR2, and HCDR3.” The CDRs contain most of the residues which form specific interactions with the antigen. The numbering and positioning of CDR amino acid residues within the LCVR and HCVR regions is in accordance with known conventions (e.g., Kabat (1991), Chothia (1987), and/or North (2011)). In different embodiments of the invention, the FRs of the antibody may be identical to the human germline sequences, or may be naturally or artificially modified.

As used herein, the term “DC101” refers to a rat monoclonal antibody directed against mouse VEGFR2 that may be used in experiments as a surrogate in mice for an anti-VEGFR2 Ab, preferably ramucirumab (see, for example, Witte L., et al. Monoclonal antibodies targeting the VEGF receptor-2 (Flk1/KDR) as an anti-angiogenic therapeutic strategy. Cancer Metastasis Rev., 17: 155-161, 1998; and/or Prewett M., et al., Antivascular endothelial growth factor receptor (fetal liver kinase 1) monoclonal antibody inhibits tumor angiogenesis and growth of several mouse and human tumors. Cancer Res., 59:5209-5218, 1999).

In certain embodiments, the anti-VEGFR2 Ab and/or the C8-H241 Ab for the methods and/or uses of the present invention are altered to increase or decrease the extent to which the antibody is glycosylated. Addition or deletion of glycosylation sites to an antibody may be conveniently accomplished by altering the amino acid sequence such that one or more glycosylation sites is created or removed.

Where the anti-VEGFR2 Ab and/or the C8-H241 Ab comprise an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region (see, e.g., Wright et al. TIBTECH 15:26-32 (1997)). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, anti-VEGFR2 Ab variants and/or C8-H241 Ab variants have a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn297 (e.g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function (see, US Patent Publication Nos. US 2003/0157108 and US 2004/0093621, for example). Examples of publications related to “defucosylated” or “fucose-deficient” antibody variants include: US 2003/0157108; WO 2000/61739; WO 2001/29246; US 2003/0115614; US 2002/0164328; and Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1; and WO 2004/056312 A1), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO 2003/085107).

Unless indicated otherwise, when referring to an amino acid residue in an antibody by a number, the EU numbering system is used herein as it is conventionally used in the art (see, Kabat, et al., Sequences of Proteins of Immunological Interest, Fifth Edition, U.S. Department of Health and Human Services, NIH Publication No. 91-3242 (1991), for example).

As used herein, the term “kit” refers to a package comprising at least two separate containers, wherein a first container contains a C8-H241 Ab and a second container contains an anti-VEGFR2 Ab. As used herein, the term “kit” also refers to a package comprising at least two separate containers, wherein a first container contains C8-H241-IgG4, preferably, emibetuzumab, and a second container contains ramucirumab. A “kit” may also include instructions to administer all or a portion of the contents of these first and second containers to a cancer patient, preferably, a HCC patient, a RCC patient, a gastric cancer patient, a patient having carcinoma of the GEJ, a lung cancer patient, or a NSCLC patient. Optionally, these kits also include a third container containing a composition comprising at least one of paclitaxel, everolimus, temsirolimus, docetaxel, pemetrexed, gemcitabine, 5-fluorouracil, folinic acid, oxaliplatin, and/or an EGFR inhibitor such as erlotinib, gefitinib, afatinib, rociletinib, AZD9291, ASP8273, or HM61713.

As used herein, the terms “treating,” “to treat,” or “treatment” refers to restraining, slowing, stopping, reducing, or reversing the progression or severity of an existing symptom, disorder, condition, or disease.

As used herein, the term, “paclitaxel” refers to a natural product with the chemical name (2α, 4α, 5β, 7β, 10β, 13α)-4,10-bis(acetyloxy)-13-{[(2R,3S)-3-(benzoylamino)-2-hydroxy-3-phenylpropanoyl]oxy}-1,7-dihydroxy-9-oxo-5,20-epoxytax-11-en-2-yl benzoate having the following chemical structure:

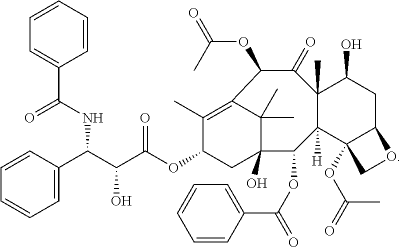 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

As used herein, the term, “5-fluorouracil” refers to the chemical name of 5-fluoro-1H,3H-pyrimidine-2,4-dione having the following chemical structure:

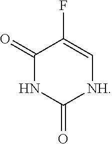

As used herein, the term, “folinic acid” refers to the chemical name of (2S)-2-{[4-[(2-amino-5-formyl-4-oxo-5,6,7,8-tetrahydro-1H-pteridin-6-yl)methylamino]benzoyl]amino}pentanedioic acid having the following chemical structure:

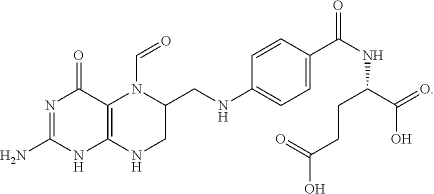 [SMILES: Nc1nc(=O)c2c([nH]1)NCC(CNc1ccc(C(=O)N[C@@H](CCC(=O)O)C(=O)O)cc1)N2C=O]

As used herein, the term, “oxaliplatin” refers to the chemical name of [(1R,2R)-cyclohexane-1,2-diamine](ethanedioato-O,O′)platinum(II) having the structure:

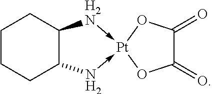

As used herein, the term, “temsirolimus” refers to an inhibitor of mammalian target of rapamycin with the chemical name of (3S,6R,1E,9R, 10R, 12R, 14S,15E, 11E, 19E, 21S, 23S, 26R, 27R,34aS)-9,10,12,13,14,21,22,23,24,25,26,27,32,33,34,34a-Hexadecahydro-9,27-dihydroxy-3-[(1R)-2-[(1S,3R,4R)-4-hydroxy-3-methoxycyclohexyl]-1-methylethyl]-10,21-dimethoxy-6,8,12,14,20,26-hexamethyl-23, 27-epoxy-3H-pyrido[2,1-c][1,4]oxaazacyclohentriacontine-1,5,11,28,29(4H,6H,31H)-pentone 4′[2,2-bis(hydroxymethyl)propionate]; or Rapamycin, 42-[3-hydroxy-2-(hydroxymethyl)-2-methylpropanoate] having the following chemical structure:

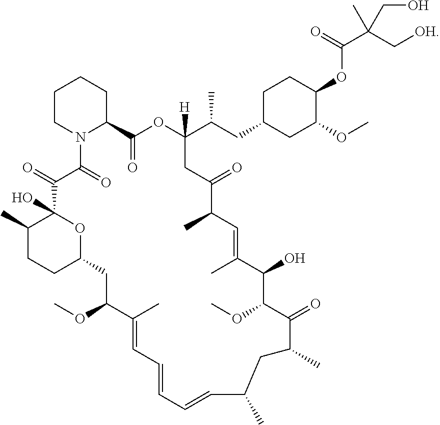 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)C(C)(CO)CO)[C@H](OC)C2)CC(=O)[C@H](C)/C=C(\C)[C@@H](O)[C@@H](OC)C(=O)[C@H](C)C[C@H](C)/C=C/C=C/C=C/1C]

As used herein, the term, “everolimus” refers to an inhibitor of mammalian target of rapamycin with the chemical name of (1R,9S,12S,15R,16E,18R,19R,21R,23S,24E,26E,28E,30S,32S,35R)-1,18-dihydroxy-12-{(1R)-2-[(1S,3R,4R)-4-(2-hydroxyethoxy)-3-methoxycyclohexyl]-1-methylethyl}-19,30-dimethoxy15,17,21,23,29,35-hexamethyl-11,36-dioxa-4-aza-tricyclo[30.3.1.0^{4,9}]hexatriaconta-16,24,26,28-tetraene-2,3,10,14,20pentaone having the following chemical structure:

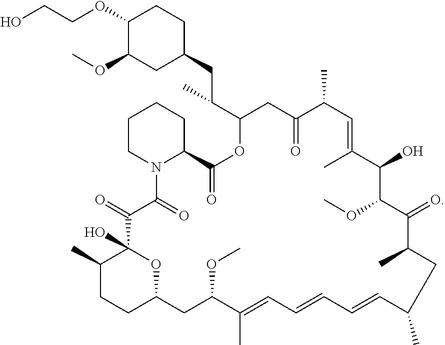 [SMILES: CO[C@H]1C[C@@H]2CC[C@@H](C)[C@@](O)(O2)C(=O)C(=O)N2CCCC[C@H]2C(=O)OC([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C]

As used herein, the term “patient” refers to a mammal, preferably a human.

As used herein, the terms “cancer” and “cancerous” refer to or describe the physiological condition in patients that is typically characterized by unregulated cell proliferation. Included in this definition are benign and malignant cancers. By “early stage cancer” or “early stage tumor” is meant a cancer that is not advanced or metastatic or is classified as a Stage 0, I, or II cancer. Examples of cancer include, but are not limited to, gastric cancer, preferably, carcinoma of the gastroesophageal junction, HCC, and RCC.

In some embodiments of the present invention, the cancer patients are selected for treatment with a combination therapy disclosed herein on the basis of having a tumor in which MET is expressed or overexpressed. Preferably, the MET expression status of a cancer patient's tumor is determined by using an immunohistochemistry (IHC) assay, PCR assay, gene sequencing assay and/or fluorescence in-situ hybridization (FISH) assay suitable for the detection of MET. More preferably, an IHC method for determining whether a cancer patient's tumor expresses or overexpresses MET is performed essentially as described in Example 7 of PCT International Publication WO 2013/169532 using an anti-MET antibody, or an antigen-binding fragment thereof, disclosed therein that specifically binds to MET-ECD, wherein said anti-MET antibody comprises a LC and a HC, wherein the amino acid sequence of the LC and HC is that given in SEQ ID NO: 22 and SEQ ID NO: 23, respectively. In some embodiments, a patient is selected for treatment with the combination therapies of the present invention after a sample of a cancer patient's tumor has been determined expressed or overexpress MET by use of an IHC assay wherein the assay comprises the step of contacting the sample with a MET antibody, or antigen-binding fragment thereof, wherein the antibody, or fragment thereof, comprises a LC and a HC, wherein the amino acid sequence of the LC and HC is that given in SEQ ID NO: 22 and SEQ ID NO: 23, respectively. In various embodiments of the methods of the present invention, the aforementioned IHC assay of the patient's tumor is performed using a formalin-fixed and paraffin-embedded sample of the patient's tumor.

An unexpected therapeutic effect of the combination treatments of the invention is the ability to produce marked anti-cancer effects in a patient without causing significant toxicities or adverse effects, so that the patient benefits from the combination treatment method overall. The efficacy, i.e., therapeutic effect(s), of the combination treatment of the invention can be measured by various endpoints commonly used in evaluating cancer treatments, including, but not limited to, tumor regression, tumor weight or size shrinkage, time to disease progression, overall survival, progression free survival, overall response rate, duration of response, and/or quality of life. The therapeutic agents used in the invention may cause inhibition of metastatic spread without shrinkage of the primary tumor, may induce shrinkage of the primary tumor, or may simply exert a tumoristatic effect. Because the invention relates to the use of a combination of unique anti-tumor agents, novel approaches to determining efficacy, i.e., therapeutic effect(s), of any particular combination therapy of the present invention can be optionally employed, including, for example, measurement of plasma or urinary markers of angiogenesis and measurement of response through radiological imaging.

As used herein, the term “Complete Response” (CR) refers to the disappearance of all target lesions. Any pathological lymph nodes (whether target or non-target) must have reduction in short axis to <10 mm.

As used herein, the term “Partial Response” (PR) refers to at least a 30% decrease in the sum of diameters of target lesions, taking as reference the baseline sum diameters.

As used herein, the term “Progressive Disease” (PD) refers to at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. For the avoidance of doubt, the appearance of one or more new lesions is also considered progression.

As used herein, the term “Stable Disease” (SD) refers to neither sufficient shrinkage to qualify for PR nor sufficient increase to qualify for PD, taking as reference the smallest sum diameters while on study.

As used herein, the term “Objective Response” (OR) refers to the sum of CR plus PR.

The skilled artisan will appreciate the terms CR, PR, PD, SD and OR correspond to definitions according to RECIST v1.1, Eisenhauer et al., European Journal of Cancer, 2009, 45, 228-247.

As used herein, the term “time to disease progression” or “TTP” refers to the time, generally measured in weeks or months, from the time of initial treatment, until the cancer progresses or worsens. Such progression can be evaluated by the skilled clinician.

As used herein, the term “extending TTP” refers to increasing the time to disease progression in a treated patient relative to i) an untreated patient or relative, or ii) a patient treated with less than all of the anti-tumor agents in a particular combination therapy.

As used herein, the term “survival” refers to the patient remaining alive, and includes overall survival as well as progression free survival.

As used herein, the term, “overall survival” refers to the patient remaining alive for a defined period of time, such as 1 year, 5 years, etc., from the time of diagnosis or treatment.

As used herein, the term, “progression free survival” refers to the patient remaining alive, without the cancer progressing or getting worse.

As used herein, the term “extending survival” is meant increasing overall or progression free survival in a treated patient relative to i) an untreated patient, ii) a patient treated with less than all of the anti-tumor agents in a particular combination therapy, or iii) a control treatment protocol. Survival is monitored for at least about one month, at least about one month, at least about two months, at least about four months, at least about six months, at least about nine months, or at least about 1 year, or at least about 2 years, or at least about 3 years, or at least about 4 years, or at least about 5 years, or at least about 10 years, etc., following the initiation of treatment or following the initial diagnosis of cancer.

As used herein, the term “primary tumor” or “primary cancer” is meant the original cancer and not a metastatic lesion located in another tissue, organ, or location in the patient's body.

As used herein, the term “effective amount” refers to the amount or dose of a C8-H241 Ab and to the amount or dose of an anti-VEGFR2 Ab which, upon single or multiple dose administration to the patient, provides an effective response in the patient under diagnosis or treatment. As used herein, the term “effective amount” also refers to the amount or dose of C8-H241-IgG4, preferably, emibetuzumab, and to the amount or dose of ramucirumab, which, upon single or multiple dose administration to the patient, provides an effective response in the patient under diagnosis or treatment. It is understood that a combination therapy of the present invention is carried out by administering a C8-H241 Ab together with an anti-VEGFR2 Ab in any manner which provides effective levels of the C8-H241 Ab and the anti-VEGFR2 Ab in the body. It is also understood that a combination therapy of the present invention is carried out by administering C8-H241-IgG4, preferably, emibetuzumab, together with ramucirumab in any manner which provides effective levels of C8-H241-IgG4, preferably, emibetuzumab, and ramucirumab in the body.

As used herein, the terms “effective response” of a patient or a patient's “responsiveness” to treatment with a combination of agents, or “therapeutic effect” refers to the clinical or therapeutic benefit(s) imparted to a patient upon administration of i) a combination of a C8-H241 Ab and an anti-VEGFR2 Ab, ii) C8-H241-IgG4 and ramucirumab, or iii) emibetuzumab and ramucirumab. Such benefit(s) include any one or more of: extending survival (including overall survival and progression free survival); resulting in an objective response (including a complete response or a partial response); tumor regression, tumor weight or size shrinkage, longer time to disease progression, increased duration of survival, longer progression free survival, improved overall response rate, increased duration of response, and improved quality of life and/or improving signs or symptoms of cancer, etc.

An effective amount can be readily determined by the attending diagnostician, as one skilled in the art, by the use of known techniques and by observing results obtained under analogous circumstances. In determining the effective amount for a patient, a number of factors are considered by the attending diagnostician, including, but not limited to: the species of patient; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

C8-H241 Ab, preferably C8-H241-IgG4, more preferably, emibetuzumab, is generally effective over a wide dosage range in the combination of the present invention. For example, dosages normally are given on days one and fifteen of a 28-day treatment cycle and each dose falls within the range of about 500 mg to about 2500 mg, preferably about 750 mg to about 2000 mg, and most preferably about 750 mg. In addition, an anti-VEGFR2 Ab, preferably ramucirumab, is generally effective over a wide dosage range in the combination of the present invention. For example, dosages per 28-day cycle normally fall within the range of about two doses (one on day one and one on day fifteen) of 6 to 10 mg/kg, preferably about 8 to about 10 mg/kg, and most preferably about 8 mg/kg. In some instances dosage levels below the lower limit of the aforesaid ranges for C8-H241-IgG4, preferably, emibetuzumab, and ramucirumab may be more than adequate, while in other cases smaller or still larger doses may be employed with acceptable side effects, and therefore the above dosage range is not intended to limit the scope of the invention in any way. When given in combination with an anti-VEGFR2 Ab, for example, over a 28-day cycle, a C8-H241 Ab, preferably, C8-H241-IgG4, more preferably, emibetuzumab, is administered on days one and fifteen within the range of about 500 mg to about 2500 mg and an anti-VEGFR2 Ab, preferably, ramucirumab, is administered on day one and day fifteen within the range of about 6 to 10 mg/kg. When given in combination, for example, over a 28-day cycle, a C8-H241 Ab, preferably C8-H241-IgG4, more preferably, emibetuzumab, is administered on day one and day fifteen within the range of about 750 mg to about 2000 mg and an anti-VEGFR2 Ab, preferably, ramucirumab, is administered on day one and day fifteen within the range of about 8 mg/kg. When given in combination, for example, over a 28-day cycle, a C8-H241 Ab, preferably, C8-H241-IgG4, preferably, emibetuzumab, is administered on day one and day fifteen within the range of about 750 mg to about 2000 mg and an anti-VEGFR2 Ab, preferably, ramucirumab, is administered on day one and day fifteen at about 8 mg/kg. Optionally, a 21-day cycle may be employed with doses given on day one with the dosage of ramucirumab within the range of about 6-10 mg/kg, more preferably, 8 mg/kg and the dosage of C8-H241-IgG4, preferably, emibetuzumab, in the range of about 750 mg to about 200 mg, more preferably, 1000 mg. If paclitaxel, everolimus, temsirolimus, erlotinib, gefitinib, afatinib, rociletinib, AZD9291, ASP8273, HM61713, or a pharmaceutically acceptable salt thereof, and/or a combination of 5-fluorouracil, folinic acid and oxaliplatin, are included in the combination, dosing should follow the approved dosages for the respective compound(s) and indication. However, one of ordinary skill in the art would know that in some instances dosage levels below the lower limit of the approved dosages may be more than adequate, while in other cases smaller or still larger doses may be employed with acceptable side effects, and therefore the approved dosages is not intended to limit the scope of the invention in any way.

A C8-H241 Ab, preferably, C8-H241-IgG4, more preferably, emibetuzumab, and an anti-VEGFR2 Ab, preferably, ramucirumab, are preferably formulated as pharmaceutical compositions administered by any route which makes the compound bioavailable. The route of administration may be varied in any way, limited by the physical properties of the drugs and the convenience of the patient and the caregiver. Preferably, an anti-VEGFR2 Ab, more preferably, ramucirumab, and a C8-H241 Ab, preferably, C8-H241-IgG4, more preferably, emibetuzumab, compositions are for parenteral administration, such as intravenous or subcutaneous administration. More preferably, C8-H241 Ab, even more preferably, C8-H241-IgG4, even more preferably, emibetuzumab, compositions comprise about 10 to about 20 mg/ml of C8-H241 Ab, preferably, C8-H241-IgG4, more preferably, emibetuzumab, about 10 to about 20 mM histidine buffer, pH 5.5-6.0, about 75 mM to about 150 mM sodium chloride, about 0.01% to about 0.06% polysorbate 80, and, optionally, about 100 mM to about 150 mM glycine. Preferably, C8-H241-IgG4, preferably, emibetuzumab, compositions are formulated for intravenous administration and comprise about 20 mg/ml of C8-H241 Ab, about 10 mM histidine buffer, pH 5.5, about 150 mM sodium chloride, and about 0.06% polysorbate 80. Such pharmaceutical compositions and processes for preparing same are well known in the art. (See, e.g., Remington: The Science and Practice of Pharmacy (D. B. Troy, Editor, 21st Edition, Lippincott, Williams & Wilkins, 2006). Paclitaxel, everolimus, temsirolimus, or a combination of 5-fluorouracil, folinic acid and oxaliplatin, are preferably formulated as pharmaceutical compositions administered by any route which makes the compound or composition bioavailable. The route of administration may be varied in any way, limited by the physical properties of the drugs and the convenience of the patient and the caregiver.

Paclitaxel is generally effective over a wide dosage range in the combination of the present invention. For example, dosages per week are normally in two doses of 90 mg/m² on the same day.

The combination of 5-fluorouracil, folinic acid and oxaliplatin, is known as FOLFOX and may be dosed according to any of the FOLFOX protocols as known by those skilled in the art. FOLFOX regimens are more commonly used by those skilled in the art for the treatment of colorectal carcinoma or gastric cancers, including, but not limited to, carcinoma of the GEJ.

For everolimus and temsirolimus, an oral 10-mg starting dose is recommended for every patient regardless of age, gender, body weight, or renal function. No premedication is required. Once-daily dose is taken at the same time every day and the drug should be taken consistently with or consistently without food. Tablets should be swallowed whole with a glass of water.

Docetaxel is generally effective over a wide dosage range in the combination of the present invention. For example, dosages per week are normally in two doses of 90 mg/m² on the same day.

An anti-VEGFR2 Ab, preferably ramucirumab, and a C8-H241 Ab, preferably C8-H241-IgG4, more preferably, emibetuzumab, may be administered simultaneously or sequentially.

As used herein, “simultaneous” administration means the administration of an anti-VEGFR2 Ab, preferably ramucirumab, and a C8-H241 Ab, preferably C8-H241-IgG4, more preferably, emibetuzumab, to the patient in a single action, which requires the two antibodies to be incorporated into a single dosage form, such as a single solution for IV administration.

As used herein, “sequential” administration means the administration of an anti-VEGFR2 Ab, preferably ramucirumab, and a C8-H241 Ab, preferably C8-H241-IgG4, more preferably, emibetuzumab, to the patient is a separate action, but the two actions are linked. For example, administering a first aqueous solution comprising ramucirumab by IV infusion and administering a second aqueous solution comprising C8-H241-IgG4, preferably, emibetuzumab, by IV infusion is considered to be sequential administration, even if the two solutions are infused into the patient at the same time or if one of the aqueous solutions is infused into the patient immediately or shortly after the infusion of the other aqueous solution. Preferably, sequential administration is the administration of an anti-VEGFR2 Ab, preferably, ramucirumab, and a C8-H241 Ab, preferably, C8-H241-IgG4, more preferably, emibetuzumab, within one, two, three, four, five, six, or seven days of each other. More preferably, sequential administration is the administration of an anti-VEGFR2 Ab, preferably, ramucirumab, and a C8-H241 Ab, preferably, C8-H241-IgG4, more preferably, emibetuzumab, within one, two, three, four, five, six, seven, eight, nine, ten, twelve, fourteen, sixteen, eighteen, twenty-one, or twenty-four hours of each other.

As used herein, the phrase “in combination with” refers to the administration of a C8-H241 Ab, preferably, emibetuzumab, with an anti-VEGFR2 Ab, preferably, ramucirumab, simultaneously. Optionally, paclitaxel, everolimus, temsirolimus, docetaxel, pemetrexed, gemcitabine, erlotinib, gefitinib, afatinib, rociletinib, AZD9291, ASP8273, HM61713 or a pharmaceutically acceptable salt thereof, and/or a combination of 5-fluorouracil, folinic acid and oxaliplatin is also administered simultaneously or sequentially.

As used herein, the phrase “in combination with” refers to the administration of C8-H241-IgG4, preferably, emibetuzumab, with ramucirumab simultaneously. Optionally, paclitaxel, everolimus, temsirolimus, docetaxel, pemetrexed, gemcitabine, erlotinib, gefitinib, afatinib, rociletinib, AZD9291, ASP8273, HM61713, or a pharmaceutically acceptable salt thereof, and/or a combination of 5-fluorouracil, folinic acid and oxaliplatin is also administered simultaneously. As used herein, the phrase “in combination with” also refers to the administration of C8-H241-IgG4, preferably, emibetuzumab, with ramucirumab sequentially in any order. Optionally, paclitaxel, everolimus, temsirolimus, docetaxel, pemetrexed, gemcitabine, erlotinib, gefitinib, afatinib, rociletinib, AZD9291, ASP8273, HM61713, or a pharmaceutically acceptable salt thereof, and/or a combination of 5-fluorouracil, folinic acid and oxaliplatin is also administered sequentially in any order. As used herein, the phrase “in combination with” also refers to the administration of C8-H241-IgG4, preferably, emibetuzumab, with ramucirumab in any combination thereof. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin is also administered in any combination thereof.

A C8-H241 Ab and an anti-VEGFR2 Ab may be administered either as part of the same pharmaceutical composition or in separate pharmaceutical compositions. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin may also be administered either as part of the same pharmaceutical composition or in separated pharmaceutical compositions. A C8-H241 Ab can be administered prior to administration of an anti-VEGFR2 Ab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered prior to administration of an anti-VEGFR2 Ab. A C8-H241 Ab can be administered at the same time as administration of an anti-VEGFR2 Ab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered at the same time as administration of an anti-VEGFR2 Ab. A C8-H241 Ab can be administered subsequent to administration of an anti-VEGFR2 Ab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered subsequent to administration of an anti-VEGFR2 Ab. A C8-H241 Ab can be administered prior to, at the same time as, or subsequent to administration of an anti-VEGFR2 Ab or in some combination thereof. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered prior to, at the same time as, or subsequent to administration of an anti-VEGFR2 Ab or in some combination thereof.

C8-H241-IgG4, preferably, emibetuzumab, and ramucirumab may be administered either as part of the same pharmaceutical composition or in separate pharmaceutical compositions. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin may also be administered either as part of the same pharmaceutical composition or in separated pharmaceutical compositions. C8-H241-IgG4, preferably, emibetuzumab, can be administered prior to administration of ramucirumab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered prior to administration of ramucirumab. C8-H241-IgG4, preferably, emibetuzumab, can be administered at the same time as administration of ramucirumab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered at the same time as administration of ramucirumab. C8-H241-IgG4, preferably, emibetuzumab, can be administered subsequent to administration of ramucirumab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered subsequent to administration of ramucirumab. C8-H241-IgG4, preferably, emibetuzumab, can be administered prior to, at the same time as, or subsequent to administration of ramucirumab or in some combination thereof. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered prior to, at the same time as, or subsequent to administration of ramucirumab or in some combination thereof.

Where an anti-VEGFR2 Ab is administered at repeated intervals (e.g. during a standard course of treatment), a C8-H241 Ab can be administered prior to each administration of an anti-VEGFR2 Ab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered prior to each administration of an anti-VEGFR2 Ab. Where an anti-VEGFR2 Ab is administered at repeated intervals (e.g. during a standard course of treatment), a C8-H241 Ab can be administered at the same time as each administration of an anti-VEGFR2 Ab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered at the same time as each administration of an anti-VEGFR2 Ab. Where an anti-VEGFR2 Ab is administered at repeated intervals (e.g. during a standard course of treatment), a C8-H241 Ab can be administered subsequent to each administration of an anti-VEGFR2 Ab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered subsequent to each administration of an anti-VEGFR2 Ab. Where an anti-VEGFR2 Ab is administered at repeated intervals (e.g. during a standard course of treatment), a C8-H241 Ab can be administered prior to, at the same time as, or subsequent to, each administration of an anti-VEGFR2 Ab or some combination thereof. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered prior to, at the same time as, or subsequent to, each administration of an anti-VEGFR2 Ab. Where an anti-VEGFR2 Ab is administered at repeated intervals (e.g. during a standard course of treatment), a C8-H241 Ab can be administered at different intervals in relation to therapy with an anti-VEGFR2 Ab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered at different intervals in relation to therapy with an anti-VEGFR2 Ab. Where an anti-VEGFR2 Ab is administered at repeated intervals (e.g. during a standard course of treatment), a C8-H241 Ab can be administered in a single or series of dose(s) prior to, at any time during, or subsequent to the course of treatment with an anti-VEGFR2 Ab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered in a single or series of dose(s) prior to, at any time during, or subsequent to the course of treatment with an anti-VEGFR2 Ab. Where an anti-VEGFR2 Ab is administered at repeated intervals (e.g. during a standard course of treatment), a C8-H241 Ab can be administered in a single dose prior to, at any time during, or subsequent to the course of treatment with an anti-VEGFR2 Ab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered in a single dose prior to, at any time during, or subsequent to the course of treatment with an anti-VEGFR2 Ab. Where an anti-VEGFR2 Ab is administered at repeated intervals (e.g. during a standard course of treatment), a C8-H241 Ab can be administered in a single dose prior to the course of treatment with an anti-VEGFR2 Ab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered prior to the course of treatment with an anti-VEGFR2 Ab. Where an anti-VEGFR2 Ab is administered at repeated intervals (e.g. during a standard course of treatment), a C8-H241 Ab can be administered in a single dose at any time during the course of treatment with an anti-VEGFR2 Ab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered in a single dose at any time during the course of treatment with an anti-VEGFR2 Ab. Where an anti-VEGFR2 Ab is administered at repeated intervals (e.g. during a standard course of treatment), a C8-H241 Ab can be administered in a single dose subsequent to the course of treatment with an anti-VEGFR2 Ab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered in a single dose subsequent to the course of treatment with an anti-VEGFR2 Ab. Where an anti-VEGFR2 Ab is administered at repeated intervals (e.g. during a standard course of treatment), a C8-H241 Ab can be administered in a series of doses prior to the course of treatment with an anti-VEGFR2 Ab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered in a series of doses prior to the course of treatment with an anti-VEGFR2 Ab. Where an anti-VEGFR2 Ab is administered at repeated intervals (e.g. during a standard course of treatment), a C8-H241 Ab can be administered in a series of doses subsequent to the course of treatment with an anti-VEGFR2 Ab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered in a series of doses subsequent to the course of treatment with an anti-VEGFR2 Ab.

Where ramucirumab is administered at repeated intervals (e.g., during a standard course of treatment), C8-H241-IgG4, preferably, emibetuzumab, can be administered prior to each administration of ramucirumab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered prior to each administration of ramucirumab. Where ramucirumab is administered at repeated intervals (e.g., during a standard course of treatment), C8-H241-IgG4, preferably, emibetuzumab, can be administered at the same time as each administration of ramucirumab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered at the same time as each administration of ramucirumab. Where ramucirumab is administered at repeated intervals (e.g. during a standard course of treatment), C8-H241-IgG4, preferably, emibetuzumab, can be administered subsequent to each administration of ramucirumab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered subsequent to each administration of ramucirumab. Where ramucirumab is administered at repeated intervals (e.g. during a standard course of treatment), C8-H241-IgG4, preferably, emibetuzumab, can be administered prior to, at the same time as, or subsequent to, each administration of ramucirumab or some combination thereof. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered prior to, at the same time as, or subsequent to, each administration of ramucirumab. Where ramucirumab is administered at repeated intervals (e.g. during a standard course of treatment), C8-H241-IgG4, preferably, emibetuzumab, can be administered at different intervals in relation to therapy with ramucirumab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered at different intervals in relation to therapy with ramucirumab. Where ramucirumab is administered at repeated intervals (e.g. during a standard course of treatment), C8-H241-IgG4, preferably, emibetuzumab, can be administered in a single or series of dose(s) prior to, at any time during, or subsequent to the course of treatment with ramucirumab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered in a single or series of dose(s) prior to, at any time during, or subsequent to the course of treatment with ramucirumab. Where ramucirumab is administered at repeated intervals (e.g. during a standard course of treatment), C8-H241-IgG4, preferably, emibetuzumab, can be administered in a single dose prior to, at any time during, or subsequent to the course of treatment with ramucirumab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered in a single dose prior to, at any time during, or subsequent to the course of treatment with ramucirumab. Where ramucirumab is administered at repeated intervals (e.g. during a standard course of treatment), C8-H241-IgG4 can be administered in a single dose prior to the course of treatment with ramucirumab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered prior to the course of treatment with ramucirumab. Where ramucirumab is administered at repeated intervals (e.g. during a standard course of treatment), C8-H241-IgG4, preferably, emibetuzumab, can be administered in a single dose at any time during the course of treatment with ramucirumab. Optionally, paclitaxel, a combination of 5-fluorouracil, folinic acid and oxaliplatin, everolimus or temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, can also be administered in a single dose at any time during the course of treatment with ramucirumab. Where ramucirumab is administered at repeated intervals (e.g. during a standard course of treatment), C8-H241-IgG4, preferably, emibetuzumab, can be administered in a single dose subsequent to the course of treatment with ramucirumab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered in a single dose subsequent to the course of treatment with ramucirumab. Where ramucirumab is administered at repeated intervals (e.g. during a standard course of treatment), C8-H241-IgG4, preferably, emibetuzumab, can be administered in a series of doses prior to the course of treatment with ramucirumab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered in a series of doses prior to the course of treatment with ramucirumab. Where ramucirumab is administered at repeated intervals (e.g. during a standard course of treatment), C8-H241-IgG4, preferably, emibetuzumab, can be administered in a series of doses subsequent to the course of treatment with ramucirumab. Optionally, paclitaxel, everolimus, temsirolimus, docetaxel, pemetrexed, gemcitabine or one or more EGFR inhibitors such as erlotinib, gefitinib, afatinib, rociletinib, AZD9291, ASP8273, HM61713, or a pharmaceutically acceptable salt thereof, and/or a combination of 5-fluorouracil, folinic acid and oxaliplatin can also be administered in a series of doses subsequent to the course of treatment with ramucirumab.

The following examples illustrate the unexpected benefit of the present combinations.

### EXAMPLE 1

### A Study of Ramucirumab in Combination with C8-11241-IgG4 or Emibetuzumab in Patients with Advanced Cancer

Study Design

This Phase 1b/2 study is a multicenter, nonrandomized, open-label, dose-escalation study to determine a recommended schedule and dose range for C8-H241-IgG4 or, preferably, emibetuzumab, that may be safely administered in combination with a fixed regimen of ramucirumab to patients with advanced and/or metastatic cancer (Part A), followed by tumor-specific expansion cohorts for gastric or GEJ adenocarcinoma, hepatocellular carcinoma, renal cell cancer, or non-small cell lung cancer patients for dose confirmation and exploration of clinical activity (Part B). During the first cycle (28 days), the dose-limiting toxicities will be assessed and potential chronic toxicity will be evaluated for the entire treatment period.

Study Objectives

The primary objective of this study is to determine a recommended schedule and dose range for C8-H241-IgG4, preferably, emibetuzumab, that may be safely administered in combination with a fixed regimen of ramucirumab to patients with advanced and/or metastatic cancer. As a co-primary objective for Part B, this study will evaluate preliminary antitumor activity observed with C8-H241-IgG4, preferably, emibetuzumab, in combination with a fixed regimen of ramucirumab, in tumor specific expansion cohorts, in terms of overall response rate (ORR).

The secondary objectives of this study are to document the antitumor activity of C8-H241-IgG4 when given in combination with ramucirumab; to characterize the safety and toxicity profile of C8-H241-IgG4, preferably, emibetuzumab, in combination with a fixed regimen of ramucirumab; to evaluate the pharmacokinetics (PK) of ramucirumab and C8-H241-IgG4, preferably, emibetuzumab, when given in combination; to document any antitumor activity observed with C8-H241-IgG4, preferably, emibetuzumab, in combination with a fixed regimen of ramucirumab; and to evaluate incidence and levels of anti-therapeutic antibodies against ramucirumab and C8-H241-IgG4, preferably, emibetuzumab, when given in combination.

The exploratory objectives of this study are to evaluate tumor tissue and blood for biomarkers related to the VEGF and MET signaling pathway and tumor biology of the respective tumor types enrolled in the study, which may include, but are not necessarily limited to, tumor expression (e.g., MET and VEGFR-2) and circulating biomarker (e.g., VEGF-A, HGF, extracellular cleaved domains of MET) and their potential association with the objectives of the study (including PK/pharmacodynamic [I′D] biomarker relationship); and to evaluate antitumor activity based on functional tumor imaging examinations, including but not limited to 2-deoxy-2[F-18]fluoro-D-glucose positron emission tomography (FDG-PET) or other relevant modalities.

Trial Drugs

Ramucirumab, provided by Lilly, is a sterile, preservative-free solution for infusion formulated in an aqueous solution at a concentration of 10 mg/mL (500 mg/50 mL vial). The buffer contains 10 mM histidine, 75 mM sodium chloride, 133 mM glycine, and 0.01% polysorbate 80. Ramucirumab is a clear or slightly opalescent and colorless or pale yellow liquid without visible particles. The pH is 6.0. The osmolality is 285 mmol/kg.

Premedication is recommended prior to infusion of ramucirumab. Recommended premedication agents include histamine H1 antagonists such as diphenhydramine hydrochloride 50 mg (or equivalent). Additional premedication may be provided at the investigator's discretion. Premedication must be provided in the setting of a prior Grade 1-2 infusion-related reaction.

Ramucirumab will be dosed at 8 mg/kg and administered as a 1-hour IV infusion prior to the administration of C8-H241-IgG4 on Days 1 and 15 of a 28-day cycle in Part A and B of this study. The infusion rate of ramucirumab should not exceed 25 mg/min.

C8-H241-IgG4, preferably, emibetuzumab, for injection is a lyophilized product supplied in glass vials containing 75 mg of C8-H241-IgG4, preferably, emibetuzumab. This product is reconstituted with 3.2 mL of sterile water for injection, resulting in 25 mg/mL of C8-H241-IgG4, preferably, emibetuzumab. The reconstituted formulation is stable for up to 6 hours at room temperature.

C8-H241-IgG4, preferably, emibetuzumab, will be administered after a minimum of a 60-minute observation period (minimum of 30-minute observation period in Cycle 2 and beyond) after the end of the ramucirumab infusion on Days 1 and 15 of a 28-day cycle as 90-minute IV infusion.

Dose levels of C8-H241-IgG4, preferably, emibetuzumab, previously tolerated as monotherapy and in combination with erlotinib will be administered following the proposed dose escalation scheme below:

Dose Level 1:

C8-H241-IgG4, preferably, emibetuzumab, 750-mg flat dose as a 90-minute IV infusion after a minimum of a 60-minute observation period (minimum of 30-minute observation period in Cycle 2 and beyond) following the end of the ramucirumab 8-mg/kg infusion on Days 1 and 15 of a 28-day cycle.

Dose Level 2:

C8-H241-IgG4, preferably, emibetuzumab, 2000-mg flat dose as a 90-minute IV infusion after a minimum of a 60-minute observation period (minimum of 30-minute observation period in Cycle 2 and beyond) following the end of the ramucirumab 8-mg/kg infusion on Days 1 and 15 of a 28-day cycle.

In Cycle 1, no dose adjustments, or delays of ramucirumab and/or C8-H241-IGG4, preferably, emibetuzumab, will be allowed (i.e., DLT assessment period), except in case of any dose limiting toxicity (DLT).

In Cycle 2 or beyond, if toxicity is experienced by a patient that warrants a dosing delay, dosing of ramucirumab and/or C8-H241-IgG4, preferably, emibetuzumab, is held for up to 2 consecutive doses (approximately 28 days) to allow sufficient time for recovery from the toxicity.

This approximate 28-day time period is to begin on the day that the next dose of study treatment should have been administered but was withheld for toxicity. If both study drugs are held due to a toxicity, study treatment with both study drugs will be resumed as soon as that toxicity resolves, provided that the patient does not meet any discontinuation criteria.

In case the toxicity is specifically attributable to ramucirumab or C8-H241-IgG4 in the opinion of the investigator (e.g., ramucirumab-related hypertension), the patient may continue to receive the other study drug following the regularly scheduled Q2W treatment time points (e.g., C8-H241-IgG4, preferably, emibetuzumab). In this setting, treatment of the other study drug (e.g, ramucirumab) is resumed at the next regularly scheduled Q2W treatment time point of the continued study drug (e.g., C8-H241-IgG4, preferably, emibetuzumab) after the event causing the hold is resolved.

Study drug infusions administered within a timing window relative to the regularly scheduled Q2W treatment time point will be considered acceptable. Moreover, in Cycle 3 or beyond, ramucirumab and C8-H241-IgG4, preferably, emibetuzumab, dosing may be delayed for up to approximately 14 days. This approximate up to 14-day time period is to begin on the day that the next dose of study treatment should have been administered. In order to keep the administration of both study drugs synchronized, the next dose of ramucirumab and C8-H241-IgG4, preferably, emibetuzumab, is administered at the same study day to continue the regularly scheduled Q2W treatment time points.

In the event of dosing delays or missed doses, disease assessment and imaging studies is undertaken according to the original study schedule, regardless of the actual number of on-study treatments received.

### EXAMPLE 2

### Efficacy Study of DC101+/− C8-H241-IgG4 in an MKN-45 Xenograft Mouse Model of Human Gastric Carcinoma

To determine the efficacy of DC101, in combination with C8-H241-IgG4, preferably, emibetuzumab, in an MKN-45 xenograft mouse model of human gastric carcinoma and to compare the combination effect to that of either monotherapy, studies conducted essentially as described below may be performed.

### Study Designs and Methods:

Obtain MKN-45 cells from the Japanese Health Resources Bank and maintain in RPMI 1640 with 10% FBS at 37° C. in 5% CO₂. Expand Cells in culture, harvest, and wash in HBSS Implant sub-confluent MKN45 cells into 60 female nu/nu mice at a concentration of 1×10⁷ cells in 0.2 ml Hank's balanced salt solution (HBSS) subcutaneously into the flank of the animal. Randomize the animals when tumors reach an average volume of 200 mm³ by tumor volume into four treatment groups (n=10):
1) Human IgG4 at 10 mg/kg, IP, once weekly (qw)
2) DC101 at 20 mg/kg, IP, 2qw
3) C8-H241-IgG4, preferably, emibetuzumab, at 10 mg/kg, IP, qw
4) DC101 at 20 mg/kg, IP, 2qw+C8-H241-IgG4 at 10 mg/kg, IP, qw
   Antibody Formulations (in PBS buffer):
   IgG4—control human IgG4 (PAA) (10.4 mg/mL)
   DC101—(11.05 mg/mL)
   C8-H241-IgG4, preferably, emibetuzumab—(14.75 mg/mL)

Administer all treatments by intraperitoneal injection (IP) starting on day 12 after tumor cell implantation when average tumor volume of 200 mm³ and ending on Day 33. Inject animals IP in a dosing volume of 10 mL/kg. Dose animals in treatment group 4, with DC101 first, followed by the C8-H241-IgG4, preferably, emibetuzumab, 30-45 minutes later.

Record tumor growth and body weight changes at least twice a week. Body weight measurement during the course of the study, is a general indicator of tolerability. Measure tumor growth with calipers and record body weights twice weekly. Calculate tumor volumes by the formula Volume (mm³)=L×W² (π/6) where L represents the larger diameter and W the smaller diameter. Calculate T/C % using the formula T/C %=100×ΔT/ΔC. Where ΔT=mean tumor volume of the drug-treated group on the final day of the study−mean tumor volume of the drug-treated group on the initial day of the dosing and ΔC=mean tumor volume of the control group on the final day of the study−mean tumor volume of the control group on the initial day of the dosing. Calculate changes in body weight by the formula (Weight on observation day−Weight on day 12)/Weight on Day 12×100. Calculate test for significant differences between treatment groups by RM ANOVA using the JMP (v.9.0.3) statistical package (SAS Institute Inc., Cary, N.C., USA).

### Results:

Treatment with DC101 or emibetuzumab, as monotherapies, significantly inhibited the growth of MKN-45 tumors (P<0.0001) with respective T/C % values of 33% and 44%.

The combination of DC101 and emibetuzumab resulted in tumor stasis. The effect was significantly greater than either monotherapy (P<0.0006) where the T/C % was 7%.

All animals survived the dosing and observation period. The only group to have a significant change in average body weight was the emibetuzumab monotherapy group (P<0.0001) which had an average weight loss of 4.1%.

DC101 (20 mg/kg, 2qw) or emibetuzumab (10 mg/kg, qw) achieved T/C % of 33% and 44%, respectively, that was statistically significant (P<0.0001) from the vehicle control group. The combination of both DC101 and emibetuzumab achieved a T/C % of 7% that was statistically significant compared to either single agent (combination as compared to DC101 P=0.0006, combination as compared to emibetuzumab P<0.0001). All mice in the vehicle control group had disease progression. Two mice out of 10 mice in each of the two monotherapy groups (DC101 or the emibetuzumab) had stable disease, and the rest of the mice in these two groups had disease progression. Seven out of the 10 mice in the combination DC101+emibetuzumab group had stable disease, 2/8 mice had disease progression and 1/10 had partial response.

To assess the effect of the combination of DC101 and emibetuzumab, a repeated measures ANOVA model was fit on log transformed tumor volume using the mixed procedure in SAS software (version 9.3, Cary, N.C.) followed by a 2×2 interaction test to test for statistical significance of the combination of the two single agents. The observed percent reduction of the combination on day 33 is 83.1%. The expected percent reduction of the combination on day 33 is 81.5%. The p-value from the 2×2 interaction test on day 33 was not significant at p=0.654. All pairwise comparisons between the combination group and each single agent group on day 33 are also statistically significant. By inspection of the means, these results indicate that the combination group is statistically smaller than each single agent group. Taking all of these results in totality the effect of combining DC101 with emibetuzumab in the MKN-45 xenograft model is additive.

SEQUENCE LISTING

<SEQ ID NO: 1; DNA; human>
GACATCCAGATGACCCAGTCTCCATCTTCCGTGTCTGCATCTATAGGAGA
CAGAGTCACCATCACTTGTCGGGCGAGTCAGGGTATTGACAACTGGTTAG
GCTGGTATCAGCAGAAACCTGGGAAAGCCCCTAAACTCCTGATCTACGAT
GCATCCAATTTGGACACAGGGGTCCCATCAAGGTTCAGTGGAAGTGGATC
TGGGACATATTTTACTCTCACCATCAGTAGCCTGCAAGCTGAAGATTTTG
CAGTTTATTTCTGTCAACAGGCTAAAGCTTTTCCTCCCACTTTCGGCGGA
GGGACCAAGGTGGACATCAAA
<SEQ ID NO: 2; PRT1; human>
DIQMTQSPSSVSASIGDRVTITCRASQGIDNWLGWYQQKPGKAPKLLIYD
ASNLDTGVPSRFSGSGSGTYFTLTISSLQAEDFAVYFCQQAKAFPPTFGG
GTKVDIK
<SEQ ID NO: 3; DNA; human>
GAGGTCCAGCTGGTGCAGTCTGGGGGAGGCCTGGTCAAGCCTGGGGGGTC
CCTGAGACTCTCCTGTGCAGCCTCTGGATTCACCTTCAGTAGCTATAGCA
TGAACTGGGTCCGCCAGGCTCCAGGGAAGGGGCTGGAGTGGGTCTCATCC
ATTAGTAGTAGTAGTAGTTACATATACTACGCAGACTCAGTGAAGGGCCG
ATTCACCATCTCCAGAGACAACGCCAAGAACTCACTGTATCTGCAAATGA
ACAGCCTGAGAGCCGAGGACACGGCTGTGTATTACTGTGCGAGAGTCACA
GATGCTTTTGATATCTGGGGCCAAGGGACAATGGTCACCGTCTCAAGC
<SEQ ID NO: 4; PRT1; human>
EVQLVQSGGGLVKPGGSLRLSCAASGFTFSSYSMNWVRQAPGKGLEWVSS
ISSSSSYIYYADSVKGRFTISRDNAKNSLYLQMNSLRAEDTAVYYCARVT
DAFDIWGQGTMVTVSS
<SEQ ID NO: 5; DNA; human>
GACATCCAGATGACCCAGTCTCCATCTTCCGTGTCTGCATCTATAGGAGA
CAGAGTCACCATCACTTGTCGGGCGAGTCAGGGTATTGACAACTGGTTAG
GCTGGTATCAGCAGAAACCTGGGAAAGCCCCTAAACTCCTGATCTACGAT
GCATCCAATTTGGACACAGGGGTCCCATCAAGGTTCAGTGGAAGTGGATC
TGGGACATATTTTACTCTCACCATCAGTAGCCTGCAAGCTGAAGATTTTG
CAGTTTATTTCTGTCAACAGGCTAAAGCTTTTCCTCCCACTTTCGGCGGA
GGGACCAAGGTGGACATCAAACGAACTGTGGCTGCACCATCTGTCTTCAT
CTTCCCGCCATCTGATGAGCAGTTGAAATCTGGAACTGCCTCTGTTGTGT
GCCTGCTGAATAACTTCTATCCCAGAGAGGCCAAAGTACAGTGGAAGGTG
GATAACGCCCTCCAATCGGGTAACTCCCAGGAGAGTGTCACAGAGCAGGA
CAGCAAGGACAGCACCTACAGCCTCAGCAGCACCCTGACGCTGAGCAAAG
CAGACTACGAGAAACACAAAGTCTACGCCTGCGAAGTCACCCATCAGGGC
CTGAGCTCGCCCGTCACAAAGAGCTTCAACAGGGGAGAGTGT
<SEQ ID NO: 6; PRT1; human>
DIQMTQSPSSVSASIGDRVTITCRASQGIDNWLGWYQQKPGKAPKLLIYD
ASNLDTGVPSRFSGSGSGTYFTLTISSLQAEDFAVYFCQQAKAFPPTFGG
GTKVDIKRTVAAPSVFIFTPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
<SEQ ID NO: 7; DNA; human>
GAGGTCCAGCTGGTGCAGTCTGGGGGAGGCCTGGTCAAGCCTGGGGGGTC
CCTGAGACTCTCCTGTGCAGCCTCTGGATTCACCTTCAGTAGCTATAGCA
TGAACTGGGTCCGCCAGGCTCCAGGGAAGGGGCTGGAGTGGGTCTCATCC
ATTAGTAGTAGTAGTAGTTACATATACTACGCAGACTCAGTGAAGGGCCG
ATTCACCATCTCCAGAGACAACGCCAAGAACTCACTGTATCTGCAAATGA
ACAGCCTGAGAGCCGAGGACACGGCTGTGTATTACTGTGCGAGAGTCACA
GATGCTTTTGATATCTGGGGCCAAGGGACAATGGTCACCGTCTCAAGCGC
TAGCACCAAGGGCCCATCGGTCCTCCCCCTGGCACCCTCCTCCAAGAGCA
CCTCTGGGGGCACAGCGGCCCTGGGCTGCCTGGTCAAGGACTACTTCCCC
GAACCGGTGACGGTGTCGTGGAACTCAGGCGCCCTGACCAGCGGCGTGCA
CACCTTCCCGGCTGTCCTACAGTCCTCAGGACTCTACTCCCTCAGCAGCG
TGGTGACCGTGCCCTCCAGCAGCTTGGGCACCCAGACCTACATCTGCAAC
GTGAATCACAAGCCCAGCAACACCAAGGTGGACAAGAGAGTTGAGCCCAA
ATCTTGTGACAAAACTCACACATGCCCACCGTGCCCAGCACCTGAACTCC
TGGGGGGACCGTCAGTCTTCCTCTTCCCCCCAAAACCCAAGGACACCCTC
ATGATCTCCCGGACCCCTGAGGTCACATGCGTGGTGGTGGACGTGAGCCA
CGAAGACCCTGAGGTCAAGTTCAACTGGTACGTGGACGGCGTGGAGGTGC
ATAATGCCAAGACAAAGCCGCGGGAGGAGCAGTACAACAGCACGTACCGT
GTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAA
CCATCTCCAAAGCCAAAGGGCAGCCCCGAGAACCACAGGTGTACACCCTG
CCCCCATCCCGGGAGGAGATGACCAAGAACCAGGTCAGCCTGACCTGCCT
GGTCAAAGGCTTCTATCCCAGCGACATCGCCGTGGAGTGGGAGAGCAATG
GGCAGCCGGAGAACAACTACAAGACCACGCCTCCCGTGCTGGACTCCGAC
GGCTCCTTCTTCCTCTATAGCAAGCTCACCGTGGACAAGAGCAGGTGGCA
GCAGGGGAACGTCTTCTCATGCTCCGTGATGCATGAGGCTCTGCACAACC
ACTACACGCAGAAGAGCCTCTCCCTGTCCCCGGGTAAA
<SEQ ID NO: 8; PRT1; human>
EVQLVQSGGGLVKPGGSLRLSCAASGFTFSSYSMNWVRQAPGKGLEWVSS
ISSSSSYIYYADSVKGRFTISRDNAKNSLYLQMNSLRAEDTAVYYCARVT
DAFDIWGQGTMVTVSSASTKGPSVLPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK
<SEQ ID NO: 9; PRT1; human>
A SVGLPSVSLD LPRLSIQKDI
LTIKANTTLQ ITCRGQRDLD WLWPNNQSGS EQRVEVTECS
DGLFCKTLTI PKVIGNDTGA YKCFYRETDL ASVIYVYVQD
YRSPFIASVS DQHGVVYITE NKNKTVVIPC LGSISNLNVS
LCARYPEKRF VPDGNRISWD SKKGFTIPSY MISYAGMVFC
EAKINDESYQ SIMYIVVVVG YRIYDVVLSP SHGIELSVGE
KLVLNCTART ELNVGIDFNW EYPSSKHQHK KLVNRDLKTQ
SGSEMKKFLS TLTIDGVTRS DQGLYTCAAS SGLMTKKNST
FVRVHEKPFV AFGSGMESLV EATVGERVRI PAKYLGYPPP
EIKWYKNGIP LESNHTIKAG HVLTIMEVSE RDTGNYTVIL
INPISKEKQS HVVSLVVYVP PQIGEKSLIS PVDSYQYGIT
QTLTCTVYAI PPPHHIHWYW QLEEECANEP SQAVSVINPY
PCEEWRSVED FQGGNKIEVN KNQFALIEGK NKTVSTLVIQ
AANVSALYKC EAVNKVGRGE RVISFHVTRG PEITLQPDMQ
PTEQESVSLW CTADRSTFEN LIWYKLGPQP LPIHVGELPT
PVCKNLDTLW KLNATMFSNS TNDILIMELK NASLQDQGDY
VCLAQDRKTK KRHCVVRQLT VLERVAPTIT GNLENQTTSI
GESIEVSCTA SGNPPPQIMW FKDNETLVED SGIVLKDGNR
NLTIRRVRKE DEGLYTCQAC SVLGCAKVEA FFIIEGAQEK
TNLEIIILVG TAVIAMFFWL LLVIILRTVK RANGGELKTG
YLSIVMDPDE LPLDEHCERL PYDASKWEFP RDRLKLGKPL
GRGAFGQVIE ADAFGIDKTA TCRTVAVKML KEGATHSEHR
ALMSELKILI HIGHHLNVVN LLGACTKPGG PLMVIVEFCK
FGNLSTYLRS KRNEFVPYKT KGARFRQGKD YVGAIPVDLK
RRLDSITSSQ SSASSGFVEE KSLSDVEEEE APEDLYKDFL
TLEHLICYSF QVAKGMEFLA SRKCIHRDLA ARNILLSEKN
VVKICDFGLA RDIYKDPDYV RKGDARLPLK WMAPETIFDR
VYTIQSDVWS FGVLLWEIFS LGASPYPGVK IDEEFCRRLK
EGIRMRAPDY TTPEMYQTML DCWHGEPSQR PIFSELVEHL
GNLLQANAQQ DGKDYIVLPI SETLSMEEDS GLSLPTSPVS
CMEEEEVCDP KFHYDNTAGI SQYLQNSKRK SRPVSVKIFE
DIPLEEPEVK VIPDDNQTDS GMVLASEELK TLEDRIKLSP
SFGGMVPSKS RESVASEGSN QTSGYQSGYH SDDTDTTVYS
SEEAELLKLI EIGVQTGSTA QILQPDSGTT LSSPPV
<SEQ ID NO: 10; DNA; Artificial Sequence>
GACATCCAGATGACCCAGTCTCCATCCTCCCTGTCTGCATCTGTAGGAGA
CAGAGTCACCATCACTTGCAGTGTCAGCTCAAGTGTATCCTCCATTTACT
TGCACTGGTATCAGCAGAAACCAGGGAAAGCCCCTAAGCTCCTGATCTAT
AGCACATCCAACTTGGCTTCTGGAGTCCCATCAAGGTTCAGTGGCAGTGG
ATCTGGGACAGATTTCACTCTCACCATCAGCAGTCTGCAACCTGAAGATT
TTGCAACTTACTACTGTCAAGTCTACAGTGGTTACCCGCTCACGTTCGGC
GGAGGGACCAAGGTGGAGATCAAA
<SEQ ID NO: 11; PRT1; Artificial Sequence>
DIQMTQSPSSLSASVGDRVTITCSVSSSVSSIYLHWYQQKPGKAPKLLIY
STSNLASGVPSRFSGSGSGTDFTLTISSLQPEDFATYYCQVYSGYPLTFG
GGTKVEIK
<SEQ ID NO: 12; DNA; Artificial Sequence>
CAGGTTCAGCTGGTGCAGTCTGGTGCTGAGGTGAAGAAGCCTGGTGCCTC
AGTGAAGGTCTCCTGCAAGGCTTCTGGTTACACCTTTACCGACTACTACA
TGCACTGGGTGCGTCAGGCCCCTGGTCAAGGTCTTGAGTGGATGGGTCGT
GTTAATCCTAACCGGAGGGGTACTACCTACAACCAGAAATTCGAGGGCCG
TGTCACCATGACCACAGACACATCCACGAGCACAGCCTACATGGAGCTGC
GTAGCCTGCGTTCTGACGACACGGCCGTGTATTACTGTGCGCGTGCGAAC
TGGCTTGACTACTGGGGCCAGGGCACCACCGTCACCGTCTCC
<SEQ ID NO: 13; PRT1; Artificial Sequence>
QVQLVQSGAEVKKPGASVKVSCKASGYTFTDYYMHWVRQAPGQGLEWMGR
VNPNRRGTTYNQKFEGRVTMTTDTSTSTAYMELRSLRSDDTAVYYCARAN
WLDYWGQGTTVTVSS
<SEQ ID NO: 14; DNA; Artificial Sequence>
GACATCCAGATGACCCAGTCTCCATCCTCCCTGTCTGCATCTGTAGGAGA
CAGAGTCACCATCACTTGCAGTGTCAGCTCAAGTGTATCCTCCATTTACT
TGCACTGGTATCAGCAGAAACCAGGGAAAGCCCCTAAGCTCCTGATCTAT
AGCACATCCAACTTGGCTTCTGGAGTCCCATCAAGGTTCAGTGGCAGTGG
ATCTGGGACAGATTTCACTCTCACCATCAGCAGTCTGCAACCTGAAGATT
TTGCAACTTACTACTGTCAAGTCTACAGTGGTTACCCGCTCACGTTCGGC
GGAGGGACCAAGGTGGAGATCAAACGAACTGTGGCTGCACCATCTGTCTT
CATCTTCCCGCCATCTGATGAGCAGTTGAAATCTGGAACTGCCTCTGTTG
TGTGCCTGCTGAATAACTTCTATCCCAGAGAGGCCAAAGTACAGTGGAAG
GTGGATAACGCCCTCCAATCGGGTAACTCCCAGGAGAGTGTCACAGAGCA
GGACAGCAAGGACAGCACCTACAGCCTCAGCAGCACCCTGACGCTGAGCA
AAGCAGACTACGAGAAACACAAAGTCTACGCCTGCGAAGTCACCCATCAG
GGCCTGAGCTCGCCCGTCACAAAGAGCTTCAACAGGGGAGAGTGCTAATA
G
<SEQ ID NO: 15; PRT1; Artificial Sequence>
DIQMTQSPSSLSASVGDRVTITCSVSSSVSSIYLHWYQQKPGKAPKLLIY
STSNLASGVPSRFSGSGSGTDFTLTISSLQPEDFATYYCQVYSGYPLTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC
<SEQ ID NO: 16; DNA; Artificial Sequence>
CAGGTTCAGCTGGTGCAGTCTGGTGCTGAGGTGAAGAAGCCTGGTGCCTC
AGTGAAGGTCTCCTGCAAGGCTTCTGGTTACACATTCACTGACTACTACA
TGCACTGGGTGCGTCAGGCCCCTGGTCAAGGTCTTGAGTGGATGGGTCGT
GTTAATCCTAACCGGAGGGGTACTACCTACAACCAGAAATTCGAGGGCCG
TGTCACCATGACCACAGACACATCCACGAGCACAGCCTACATGGAGCTGC
GTAGCCTGCGTTCTGACGACACGGCCGTGTATTACTGTGCGCGTGCGAAC
TGGCTTGACTACTGGGGCCAGGGCACCACCGTCACCGTCTCCTCCGCCTC
CACCAAGGGCCCATCGGTCTTCCCGCTAGCGCCCTGCTCCAGGAGCACCT
CCGAGAGCACAGCCGCCCTGGGCTGCCTGGTCAAGGACTACTTCCCCGAA
CCGGTGACGGTGTCGTGGAACTCAGGCGCCCTGACCAGCGGCGTGCACAC
CTTCCCGGCTGTCCTACAGTCCTCAGGACTCTACTCCCTCAGCAGCGTGG
TGACCGTGCCCTCCAGCAGCTTGGGCACGAAGACCTACACCTGCAACGTA
GATCACAAGCCCAGCAACACCAAGGTGGACAAGAGAGTTGAGTCCAAATA
TGGTCCCCCATGCCCACCCTGCCCAGCACCTGAGGCCGCCGGGGGACCAT
CAGTCTTCCTGTTCCCCCCAAAACCCAAGGACACTCTCATGATCTCCCGG
ACCCCTGAGGTCACGTGCGTGGTGGTGGACGTGAGCCAGGAAGACCCCGA
GGTCCAGTTCAACTGGTACGTGGATGGCGTGGAGGTGCATAATGCCAAGA
CAAAGCCGCGGGAGGAGCAGTTCAACAGCACGTACCGTGTGGTCAGCGTC
CTCACCGTCCTGCACCAGGACTGGCTGAACGGCAAGGAGTACAAGTGCAA
GGTCTCCAACAAAGGCCTCCCGTCCTCCATCGAGAAAACCATCTCCAAAG
CCAAAGGGCAGCCCCGAGAGCCACAGGTGTACACCCTGCCCCCATCCCAG
GAGGAGATGACCAAGAACCAGGTCAGCCTGACCTGCCTGGTCAAAGGCTT
CTACCCCAGCGACATCGCCGTGGAGTGGGAAAGCAATGGGCAGCCGGAGA
ACAACTACAAGACCACGCCTCCCGTGCTGGACTCCGACGGCTCCTTCTTC
CTCTACAGCAGGCTAACCGTGGACAAGAGCAGGTGGCAGGAGGGGAATGT
CTTCTCATGCTCCGTGATGCATGAGGCTCTGCACAACCACTACACACAGA
AGAGCCTCTCCCTGTCTCTGGGT
<SEQ ID NO: 17; PRT1; Artificial Sequence>
QVQLVQSGAEVKKPGASVKVSCKASGYTFTDYYMHWVRQAPGQGLEWMGR
VNPNRRGTTYNQKFEGRVTMTTDTSTSTAYMELRSLRSDDTAVYYCARAN
WLDYWGQGTTVTVSSASTKGPSVFPLAPCSRSTSESTAALGCLVKDYFPE
PVTVSWNSGALTSGVHTFPAVLQSSGLYSLSSVVTVPSSSLGTKTYTCNV
DHKPSNTKVDKRVESKYGPPCPPCPAPEAAGGPSVFLFPPKPKDTLMISR
TPEVTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFF
LYSRLTVDKSRWQEGNVFSCSVMHEALHNHYTQKSLSLSLG
<SEQ ID NO: 18; PRT; Homo sapiens>
MKAPAVLAPGILVLLFTLVQRSNGECKEALAKSEMNVNMKYQLPNFTAET
PIQNVILHEHHIFLGATNYIYVLNEEDLQKVAEYKTGPVLEHPDCFPCQD
CSSKANLSGGVWKDNINMALVVDTYYDDQLISCGSVNRGTCQRHVFPHNH
TADIQSEVHCIFSPQIEEPSQCPDCVVSALGAKVLSSVKDRFINFFVGNT
INSSYFPDHPLHSISVRRLKETKDGFMFLTDQSYIDVLPEFRDSYPIKYV
HAFESNNFIYFLTVQRETLDAQTFHTRIIRFCSINSGLHSYMEMPLECIL
TEKRKKRSTKKEVFNILQAAYVSKPGAQLARQIGASLNDDILFGVFAQSK
PDSAEPMDRSAMCAFPIKYVNDFFNKIVNKNNVRCLQHFYGPNHEHCFNR
TLLRNSSGCEARRDEYRTEFTTALQRVDLFMGQFSEVLLTSISTFIKGDL
TIANLGTSEGRFMQVVVSRSGPSTPHVNFLLDSHPVSPEVIVEHTLNQNG
YTLVITGKKITKIPLNGLGCRHFQSCSQCLSAPPFVQCGWCHDKCVRSEE
CLSGTWTQQICLPAIYKVFPNSAPLEGGTRLTICGWDFGFRRNNKFDLKK
TRVLLGNESCTLTLSESTMNTLKCTVGPAMNKHFNMSIIISNGHGTTQYS
TFSYVDPVITSISPKYGPMAGGTLLTLTGNYLNSGNSRHISIGGKTCTLK
SVSNSILECYTPAQTISTEFAVKLKIDLANRETSIFSYREDPIVYEIHPT
KSFISGGSTITGVGKNLNSVSVPRMVINVHEAGRNFTVACQHRSNSEIIC
CTTPSLQQLNLQLPLKTKAFFMLDGILSKYFDLIYVHNPVFKPFEKPVMI
SMGNENVLEIKGNDIDPEAVKGEVLKVGNKSCENIHLHSEAVLCTVPNDL
LKLNSELNIEWKQAISSTVLGKVIVQPDQNFT
<SEQ ID NO: 19; PRT; Homo sapiens>
ECKEALAKSEMNVNMKYQLPNFTAETPIQNVILHEHHIFLGATNYIYVLN
EEDLQKVAEYKTGPVLEHPDCFPCQDCSSKANLSGGVWKDNINMALVVDT
YYDDQLISCGSVNRGTCQRHVFPHNHTADIQSEVHCIFSPQIEEPSQCPD
CVVSALGAKVLSSVKDRFINFFVGNTINSSYFPDHPLHSISVRRLKETKD
GFMFLTDQSYIDVLPEFRDSYPIKYVHAFESNNFIYFLTVQRETLDAQTF
HTRIIRFCSINSGLHSYMEMPLECILTEKRKKRSTKKEVFNILQAAYVSK
PGAQLARQIGASLNDDILFGVFAQSKPDSAEPMDRSAMCAFPIKYVNDFF
NKIVNKNNVRCLQHFYGPNHEHCFNRTLLRNSSGCEARRDEYRTEFTTAL
QRVDLFMGQFSEVLLTSISTFIKGDLTIANLGTSEGRFMQVVVSRSGPST
PHVNFLLDSHPVSPEVIVEHTLNQNGYTLVITGKKITKIPLNGLGCRHFQ
SCSQCLSAPPFVQCGWCHDKCVRSEECLSGTWTQQICLPAIYKVFPNSAP
LEGGTRLTICGWDFGFRRNNKFDLKKTRVLLGNESCTLTLSESTMNTLKC
TVGPAMNKHFNMSIIISNGHGTTQYSTFSYVDPVITSISPKYGPMAGGTL
LTLTGNYLNSGNSRHISIGGKTCTLKSVSNSILECYTPAQTISTEFAVKL
KIDLANRETSIFSYREDPIVYEIHPTKSFISGGSTITGVGKNLNSVSVPR
MVINVHEAGRNFTVACQHRSNSEIICCTTPSLQQLNLQLPLKTKAFFMLD
GILSKYFDLIYVHNPVFKPFEKPVMISMGNENVLEIKGNDIDPEAVKGEV
LKVGNKSCENIHLHSEAVLCTVPNDLLKLNSELNIEWKQAISSTVLGKVI
VQPDQNFT
<SEQ ID NO: 20; PRT; Homo sapiens>
ECKEALAKSEMNVNMKYQLPNFTAETPIQNVILHEHHIFLGATNYIYVLN
EEDLQKVAEYKTGPVLEHPDCFPCQDCSSKANLSGGVWKDNINMALVVDT
YYDDQLISCGSVNRGTCQRHVFPHNHTADIQSEVHCIFSPQIEEPSQCPD
CVVSALGAKVLSSVKDRFINFFVGNTINSSYFPDHPLHSISVRRLKETKD
GFMFLTDQSYIDVLPEFRDSYPIKYVHAFESNNFIYFLTVQRETLDAQTF
HTRIIRFCSINSGLHSYMEMPLECILTEKRKKR
<SEQ ID NO: 21; PRT; Homo sapiens>
STKKEVFNILQAAYVSKPGAQLARQIGASLNDDILFGVFAQSKPDSAEPM
DRSAMCAFPIKYVNDFFNKIVNKNNVRCLQHFYGPNHEHCFNRTLLRNSS
GCEARRDEYRTEFTTALQRVDLFMGQFSEVLLTSISTFIKGDLTIANLGT
SEGRFMQVVVSRSGPSTPHVNFLLDSHPVSPEVIVEHTLNQNGYTLVITG
KKITKIPLNGLG
<SEQ ID NO: 22; PRT; Artificial Sequence>
EIQMTQSPASLSASVGETVTITCRASENIYSYLAWYQRKQGRSPQLLVYN
AKPLAEGVPSRFSGSGSGTQFSLKINSLQPEDFGTYYCQHHYGTPFTFGS
GTRLEIKRADAAPTVSIFPPSSEQLTSGGASVVCFLNNFYPKDINVKWKI
DGSERQNGVLNSWTDQDSKDSTYSMSSTLTLTKDEYERHNSYTCEATHKT
STSPIVKSFNRNEC
<SEQ ID NO: 23; PRT; Artificial Sequence>
EVQLQQSGTVLARPGASVKMSCKASGYSFTSYWMYWVKQRPGQGLEWIGG
FHPRNSGTNYNQKFKGKAKLTAVTSASTAYMELSSLTNEDSAVYYCTRGY
YYDGSFTYWGQGTLVTVSAAKTTPPSVYPLAPGSAAQTNSMVTLGCLVKG
YFPEPVTVTWNSGSLSSGVHTFPAVLQSDLYTLSSSVTVPSSTWPSETVT
CNVAHPASSTKVDKKIVPRDCGCKPCICTVPEVSSVFIFPPKPKDVLTIT
LTPKVTCVVVDISKDDPEVQFSWFVDDVEVHTAQTQPREEQFNSTFRSVS
ELPIMHQDWLNGKEFKCRVNSAAFPAPIEKTISKTKGRPKAPQVYTIPPP
KEQMAKDKVSLTCMITDFFPEDITVEWQWNGQPAENYKNTQPIMDTDGSY
FVYSKLNVQKSNWEAGNTFTCSVLHEGLHNHHTEKSLSHSPGK

We claim:

1.-54. (canceled)

55. A method of treating cancer in a patient, comprising administering to a cancer patient in need of such treatment an effective amount of a first antibody comprising a light chain (LC) whose amino acid sequence is that given in SEQ ID NO: 15, and a heavy chain (HC) whose amino acid sequence is that given in SEQ ID NO: 17, wherein the first antibody specifically binds to MET-ECD in combination with an effective amount of a second antibody comprising a light chain variable region (LCVR) whose amino acid sequence is that given in SEQ ID NO: 2, and a heavy chain variable region (HCVR) whose amino acid sequence is that given in SEQ ID NO: 4, wherein the second antibody specifically binds to VEGFR2.

56. The method according to claim 55, wherein the first antibody is emibetuzumab.

57. The method according to claim 56 wherein the second antibody comprises a LC whose amino acid sequence is that given in SEQ ID NO: 6, and a HC whose amino acid sequence is that given in SEQ ID NO: 8.

58. The method according to claim 57, wherein the second antibody is ramucirumab.

59. The method according to 58 wherein the first antibody is administered at a dose of between about 500 mg to about 2500 mg once every two weeks and the second antibody is administered once every two weeks at a dose of between about 6 mg/kg to about 10 mg/kg.

60. The method according to claim 59, wherein the cancer is gastric, carcinoma of the gastroesophageal junction (GEJ), or hepatocellular carcinoma (HCC).

61. The method according to claim 60, wherein paclitaxel, a combination of 5-fluorouracil, folinic acid and oxaliplatin, or a pharmaceutically acceptable salt thereof, is also administered.

62. The method according to claim 59, wherein the cancer is RCC.

63. The method according to claim 62, wherein everolimus, temsirolimus, or a pharmaceutically acceptable salt thereof, is also administered.

64. The method according to claim 59, wherein the cancer is NSCLC.

65. The method according to claim 64, wherein docetaxel, pemetrexed, gemcitabine, erlotinib, gefitinib, afatinib, rociletinib, AZD9291, ASP8273, HM61713, or a pharmaceutically acceptable salt thereof, is also administered.

66. A kit comprising a first antibody comprising a light chain (LC) whose amino acid sequence is that given in SEQ ID NO: 15, and a heavy chain (HC) whose amino acid sequence is that given in SEQ ID NO: 17, wherein the first antibody specifically binds to MET-ECD in combination with an effective amount of a second antibody comprising a light chain variable region (LCVR) whose amino acid sequence is that given in SEQ ID NO: 2, and a heavy chain variable region (HCVR) whose amino acid sequence is that given in SEQ ID NO: 4, wherein the second antibody specifically binds to VEGFR2.

67. The kit according to claim 66 wherein the first antibody is emibetuzumab.

68. The kit according to claim 67 wherein the second antibody comprises a LC whose amino acid sequence is that given in SEQ ID NO: 6, and a HC whose amino acid sequence is that given in SEQ ID NO: 8.

69. The kit according to claim 68 wherein the second antibody is ramucirumab.

70. The kit according to claim 69 wherein the kit further comprises a composition comprising at least one of paclitaxel, everolimus, temsirolimus, docetaxel, pemetrexed, gemcitabine, 5-fluorouracil, folinic acid, oxaliplatin, erlotinib, gefitinib, afatinib, rociletinib, AZD9291, ASP8273, HM61713, or a pharmaceutically acceptable salt thereof.

71. A kit, comprising a pharmaceutical composition, comprising emibetuzumab, with one or more pharmaceutically acceptable carriers, diluents, or excipients, and a pharmaceutical composition, comprising ramucirumab, with one or more pharmaceutically acceptable carriers, diluents, or excipients.

72. The method according to claim 59 wherein a sample of the patient's tumor has been determined to express or overexpress MET by use of an IHC assay wherein the assay comprises the step of contacting a sample of the patient's tumor with a MET antibody, or antigen-binding fragment thereof, wherein the antibody, or antigen-binding fragment thereof, comprises a LC and a HC, wherein the amino acid sequence of the LC and HC is that given in SEQ ID NO: 22 and SEQ ID NO: 23, respectively.

73. The method according to claim 60 wherein a sample of the patient's tumor has been determined to express or overexpress MET by use of an IHC assay wherein the assay comprises the step of contacting a sample of the patient's tumor with a MET antibody, or antigen-binding fragment thereof, wherein the antibody, or antigen-binding fragment thereof, comprises a LC and a HC, wherein the amino acid sequence of the LC and HC is that given in SEQ ID NO: 22 and SEQ ID NO: 23, respectively.
